(19) United States
(12) Patent Application Publication
(10) Pub. No.: US 2026/0056187 A1
(43) Pub. Date: Feb. 26, 2026

(54) METHODS OF FORMING PATIENT-DERIVED 3D CELL CULTURES FOR TRACKING LIVE IMMUNE-TUMOR INTERACTIONS

(71) Applicant: OUROTECH, INC., Dover, DE (US)

(72) Inventors: Jay KEARNEY, London (GB); Gastón Agustín PRIMO, London (GB); Eleonora PEERANI, London (GB); Duleeka RANATUNGA, London (GB); Thomas David Laurent RICHARDSON, London (GB); Keqian NAN, London (GB); Francesco IORI, London (GB)

(21) Appl. No.: 19/103,760

(22) PCT Filed: Aug. 16, 2023
(86) PCT No.: PCT/US2023/030387

§ 371 Date: Feb. 13, 2025

Related U.S. Application Data
(60) Provisional application No. 63/398,279, filed on Aug. 16, 2022.
(60) Provisional application No. 63/470,379, filed on Jun. 1, 2023.
(60) Provisional application No. 63/470,691, filed on Jun. 2, 2023.
(60) Provisional application No. 63/471,820, filed on Jun. 8, 2023.

(51) Int. Cl. G01N 33/50; C12N 5/078; G01N 1/30; G01N 33/68

(52) U.S. Cl. CPC G01N 33/5082; C12N 5/0634; G01N 1/30; G01N 33/5011; G01N 33/5029; G01N 33/5032; G01N 33/6863; C12N 2503/02; C12N 2513/00; C12N 2539/00; G01N 2333/475; G01N 2333/521; G01N 2500/10

(57) ABSTRACT

A method of forming a three-dimensional cell culture includes obtaining a tissue sample and a matched blood sample from a patient, isolating and staining tissue-derived cells, isolating and staining immune cells, culturing the tissue-derived cells in a hydrogel, and adding at least a portion of the immune cells to the hydrogel less than 24 hours later. The method mimics an in vivo tumor-immune environment and permits analysis of tumor-immune, immune-immune, and tumor-tumor cell interactions.

### CROSS-REFERENCE TO RELATED APPLICATIONS

This application claims priority to U.S. Provisional Patent Application Nos. 63/398,279, filed Aug. 16, 2022, entitled “Method of Forming Patient-Derived 3D Cell Cultures for Tracking Live Immune-Cancer Interactions,” U.S. Provisional Patent Application No. 63/470,399, filed Jun. 1, 2023, entitled “Method of Forming Patient-Derived 3D Cell Cultures for Tracking Live Immune-Cancer Interactions,” U.S. Provisional Patent Application No. 63/470,691, filed Jun. 2, 2023, entitled “Method of Forming Patient-Derived 3D Cell Cultures for Tracking Live Immune-Cancer Interactions,” and U.S. Provisional Patent Application No. 63/471,820, filed Jun. 8, 2023, entitled “A Method of Forming Patient-Derived 3D Cell Cultures for Tracking Live Immune-Cancer Interactions,” each of which is incorporated herein by reference in the entirety and for all purposes.

### TECHNICAL FIELD

The present disclosure relates generally to three-dimensional cell cultures derived from a tissue sample, such as a tumor sample, and matched whole blood.

### BACKGROUND

A range of monolayer (two-dimensional) cell culture models have been developed to study immune cell interactions with cancer cells. These models, being cost effective, high-throughput, and standardized, have provided insight into the mechanisms of cancer immunosurveillance and immune evasion. However, they fall short on recapitulating the high complexity of in vivo scenarios due to their reductionist approach to cell-cell and cell-microenvironment interactions. Animal models remain the gold standard in preclinical cancer research. Nevertheless, the limited predictive ability of animal models in immuno-oncology is reflected in clinical results, where issues regarding drug safety, efficacy, and lack of human-specific biomarkers are increasing. There remains a need for 3D cell culture models that are able to quickly and accurately recreate the native human disease, incorporating both patient tumor and immune cells to track tumor cell migration, immune cell activation, immune cell infiltration, immune cell killing, immune evasion mechanisms, and other tumor-immune interactions in real-time.

### SUMMARY

The present disclosure includes novel methods of forming three-dimensional tumor-immune cell cultures from a tumor and matched whole blood that mimic an in vivo tumor-immune environment. The cultures are prepared and commenced quickly, which helps the constituent cells maintain their native functionality. The disclosure also includes methods of imaging and analyzing features of and interactions in the 3D culture at a single-cell and single-event level within the context of the entire 3D culture and over time, such as over hours, days, or weeks. The disclosed culturing and monitoring methods may be used to test the efficacy of potential therapeutics ex vivo, discover predictive biomarkers for patient stratification, and develop novel therapies.

In accordance with specific embodiments of the present disclosure, a method of forming and monitoring a three-dimensional cell culture may involve obtaining a tissue sample and a blood sample from a patient, staining isolated tissue-derived cells from the tissue sample, staining immune cells from peripheral blood mononuclear cells (PBMCs) isolated from the blood sample, combining the tissue-derived cells and a hydrogel to form a cell-containing hydrogel, commencing, within one hour of staining the isolated tissue-derived cells, to culture the cell-containing hydrogel, adding, within 24 hours of commencing to culture, the immune cells to the hydrogel to form a 3D immune-tissue cell culture, adding a test agent, and monitoring the cell culture over time by measuring at least two effects of the test agent on the cell culture, the effects selected from number of the immune cells, death of the immune cells, interactions between immune cells, immune cell infiltration of the cell-containing hydrogel, immune cell engagement of the tissue-derived cells, immune cell killing of the tissue-derived cells, immune cell serial killing of the tissue-derived cells, death of the tissue-derived cells, and exhaustion of the immune cells. The effects may be measurable within 48 hours of adding the test agent. The tissue may be selected from either or both of a tumor and healthy tissue.

In some embodiments of the method, the monitoring is performed by live-cell microscopy such as confocal, widefield, lightsheet, or multi-photon microscopy. In some embodiments, the monitoring includes measuring at least one of dye fluorescence from the immune cell, dye fluorescence from the tissue-derived cell, pixel or voxel size of the immune cell, pixel or voxel size of the tissue-derived cell, pixel or voxel size of a group of immune and/or tissue-derived cells, xyz location coordinates of the immune cell, xyz location coordinates of the tissue-derived cell, speed of the immune cell, speed of the tissue-derived cell, velocity of the immune cell, and velocity of the tissue-derived cell.

In some embodiments, the measuring is performed while maintaining the immune-tissue cell culture as an intact 3D immune-tissue cell culture. In some embodiments, the 3D immune-tissue cell culture is viable for up to 14 days. In some embodiments, the measuring is performed while preserving the viability of the 3D immune-tissue cell culture. In some embodiments, the 3D immune-tissue cell culture is not damaged or inactivated by the measuring.

In some embodiments, measuring immune cell infiltration includes counting a number of the immune cells within the tissue-derived cell-containing hydrogel. In some embodiments, measuring immune cell infiltration includes calculating a distance in at least one of the x, y, and z direction traveled by the immune cells over time. In some embodiments, measuring engagement of the tissue-derived cells includes counting tissue-immune cell contact events. In some embodiments, measuring serial killing of the tissue-derived cells includes counting tissue-derived cell death events. In some embodiments, measuring exhaustion of the immune cells includes calculating a speed traveled by the immune cells. In some embodiments, measuring exhaustion of the immune cells includes measuring a level of at least one soluble factor. The soluble factor may be a cytokine, chemokine, or growth factor. In some embodiments, measuring death of the immune cells includes counting the number of instances of co-localization between the immune cells and a dye that stains dead cells. In some embodiments, measuring interactions between immune cells includes counting the number of instances of contact between at least two immune cells.

In some embodiments, a difference between at least one of the at least two effects of the test agent on the 3D immune-tissue cell culture and the same one of the at least two effects of a control agent on the 3D immune-tissue cell culture is quantifiable within 48 hours of adding the test agent. The difference may be statistically significant difference.

In some embodiments, the at least one effect is immune cell infiltration of the tissue-derived cell-containing hydrogel and the difference is quantifiable without disrupting the 3D immune-tissue cell culture. In some embodiments, the at least one effect is immune cell infiltration of the tissue-derived cell-containing hydrogel and the difference is quantifiable at least twice as quickly as measuring immune cell infiltration in an animal model of cancer comprising immunodeficient mice reconstituted with a human immune system. In some embodiments, the at least one effect is immune cell engagement of the tissue-derived cells and the difference is quantifiable without disrupting the 3D immune-tissue cell culture. In some embodiments, the at least one effect is immune cell engagement of the tissue-derived cells and the difference is quantifiable at least twice as quickly as measuring immune cell engagement of the tissue-derived cells in an animal model of cancer comprising immunodeficient mice reconstituted with a human immune system. In some embodiments, the at least one effect is immune cell killing of the tissue-derived cells and the difference is quantifiable without disrupting the 3D immune-tissue cell culture. In some embodiments, the at least one effect is immune cell serial killing of the tissue-derived cells and the difference is quantifiable without disrupting the 3D immune-tissue cell culture. In some embodiments, the at least one effect is immune cell serial killing of the tissue-derived cells and the difference is quantifiable at least twice as quickly as measuring immune cell serial killing of the tissue-derived cells in an animal model of cancer comprising immunodeficient mice reconstituted with a human immune system. In some embodiments, the at least one effect is exhaustion of the immune cells and the difference is quantifiable without disrupting the 3D immune-tissue cell culture.

In some embodiments, the measuring is performed on each immune cell and each tissue-derived cell.

In some embodiments, the immune cells include at least a first immune cell fraction and a second immune cell fraction, and the first immune cell fraction is stained with a dye that produces a color different from the second immune cell fraction. At least the first immune cell fraction or the second immune cell fraction may be activated, and the activation may be done by exposing the cells to at least one of a T cell activating agent, a lipopolysaccharide, a cytokine, or a colony stimulating factor. At least the first immune cell fraction or the second immune cell fraction may include at least one of CD8 positive cells, CD14 positive cells, and CD56 positive cells. At least the first immune cell fraction or the second immune cell fraction may include CD8 negative cells.

In some embodiments, the tissue-derived cells are stained with a cell membrane permeable dye. They dye may stain at least one of lipids, proteins, organelles, cytoplasm, nuclei, and DNA. In some embodiments, the tissue-derived cells are stained with a cell membrane impermeable dye. The dye may stain DNA.

In some embodiments, the tissue sample is obtained from a tumor of the breast, kidney, liver, brain, ovary, pancreas, lung, colon, bladder, or stomach, or a metastasis of such a tumor, or from healthy tissue adjacent the tumor.

In some embodiments, the test agent is selected from a small molecule therapeutic, a large molecule therapeutic, a soluble immunosuppressive-signaling inhibitor, a checkpoint inhibitor, an immune activator, a virus, a bacteria, a gene therapy, and a cell therapy. The cell therapy may be lymphocyte-based therapy or myeloid-based therapy. Lymphocyte-based therapy may be a T-cell receptor therapy or a chimeric antigen receptor (CAR) T-cell therapy,

In some embodiments, the cell culture includes a ratio of from 1 tissue to 1 immune cell to 1 tissue to 100 immune cells. In some embodiments, the immune cells are added in a solid or liquid medium around the hydrogel. In some embodiments, the immune cells are added in a suspension to an exposed surface of the hydrogel.

The method may further include terminating the culture and running an endpoint assay or extracting at least one of DNA, RNA, and proteins.

This Summary is neither intended as, nor should it be construed as, being representative of the full extent and scope of the present disclosure. Moreover, references made herein to “the present disclosure,” or aspects thereof, should be understood to mean certain embodiments of the present disclosure and should not necessarily be construed as limiting all embodiments to a particular description. The present disclosure is set forth in various levels of detail in this Summary as well as in the attached drawings and Detailed Description, and no limitation as to the scope of the present disclosure is intended by either the inclusion or non-inclusion of elements, components, etc. in this Summary. Features from any of the disclosed embodiments may be used in combination with one another, without limitation. In addition, other features and advantages of the present disclosure will become apparent to those of ordinary skill in the art through consideration of the following Detailed Description and the accompanying drawings.

### BRIEF DESCRIPTION OF THE DRAWINGS

The drawings illustrate several embodiments of the invention, wherein identical reference numerals refer to identical or similar elements or features in different views or embodiments shown in the drawings. All microscopy images were obtained with a confocal microscope unless otherwise indicated.

FIG. 1 is a flow chart of a method according to at least one embodiment of the present disclosure.

FIG. 2 is a flow chart of a method according to at least one embodiment of the present disclosure.

FIG. 3 is a flow chart of a method according to at least one embodiment of the present disclosure.

FIGS. 4A & 4B are maximum intensity projections of a tumor cell culture before (FIG. 4A) and after (FIG. 4B) the application of a median denoising filter.

FIG. 5A shows a zoomed-in maximum intensity projection of the edge of a tumor cell culture that has been denoised; FIG. 5B shows the background image; and FIG. 5C shows the image after background subtraction.

FIGS. 6A-6C show zoomed-in maximum intensity projections of the edge of a tumor culture. FIG. 6A shows a binary image obtained after thresholding the pre-processed image;

FIG. 6B shows detected blob-like objects detected using a blob detection algorithm; and FIG. 6C shows the segmented cells obtained from watershed segmentation.

FIGS. 7A & 7B show maximum intensity projections of tumor-immune culture images with their corresponding cell detections represented as spheres.

FIG. 8A shows an image of tumor cells and corresponding detections. FIG. 8B shows an image of dead cells and corresponding detections. FIG. 8C shows the two dyes co-localize in two cells.

FIG. 9A is a maximum intensity projection of a tumor-immune culture showing tumor cells, CD8+, CD8″, and dead cells. FIG. 9B indicates cell counts from the associated imaging channels.

FIG. 10A shows an image of CD8 immune cells and corresponding detections. FIG. 10B shows dead cells and corresponding detections. FIG. 10C shows the two dyes co-localize in one cell.

FIGS. 11A-11C are 3D fluorescent readout images of tumor samples after cell isolation and within 3D hydrogel cultures. FIG. 11D is a pie chart showing the distribution of various cell types obtained from quantification of 3D fluorescent images within the tumor samples. FIG. 11E shows flow cytometry analyses of isolated cells.

FIG. 12 is an image of cells stained with a live cell dye after isolation from a tumor sample.

FIG. 13 is a line graph showing optical density of a crosslinking hydrogel over time.

FIGS. 14A & 14B shows histograms indicating the effect of patient PBMC CD3 activation on CD95 T cell expression.

FIGS. 15A-15C are maximum intensity projections of 10,000 tumor cells encapsulated in hydrogels and cultured on their own (FIG. 15A), with 50,000 PBMCs (FIGS. 15B & 15D), or with 100,000 PBMCs (FIG. 15C).

FIGS. 16A-16D are maximum intensity projections of tumor cells encapsulated in hydrogels and stained with Constituent A (FIG. 16A), Constituent B (FIG. 16B), Constituent C (FIG. 16C), or all three constituents together (FIG. 16D).

FIG. 17A is a maximum intensity projection of tumor cells, CD8+, and CD8 cells in a tumor-immune hydrogel culture. Corresponding cell counts are shown in the table of FIG. 17B.

FIG. 18 is a 3D volume rendering view of an immune-tumor co-culture.

FIG. 19 shows images from a live cell time-lapse recorded at 1-hour intervals over 16 hours.

FIG. 20 is a maximum intensity projection of a tumor-immune hydrogel culture over four days.

FIG. 21 is a bar graph showing supernatant TNFα levels in PBMC-hydrogel cultures treated with a receptor tyrosine kinase inhibitor or control for each day of a three-day culture.

FIGS. 22A-22C shows an RNAseq analysis of transcripts from patients' 3D tumor cell cultures (Day=3) compared to cell pellets, including a principal component analysis (FIG. 22A) of mRNA transcripts, a volcano plot (FIG. 22B) of differentially expressed genes, and a gene ontology analysis (FIG. 22C).

FIG. 23A is a one-hour time-lapse of migrating CD8+ cells, and FIG. 23B is of tumor cells.

FIG. 24A is a confocal microscopy image of a tumor-immune co-culture showing the interface between the hydrogel and culture medium. FIG. 24B shows the total detected CD8-immune cells and FIG. 24C shows the CD8-immune cells that infiltrated the hydrogel. Associated cell counts are shown in FIG. 24D.

FIG. 25A shows a maximum intensity projection of CD8⁻ immune cells and dead cells. FIG. 25B is a chart showing cell counts of CD8-cells, all dead cells, and dead CD8⁻ cells, obtained by dye co-localization.

FIG. 26A shows a maximum intensity projection of tumor cells and dead cells. FIG. 26B is a chart showing cell counts of tumor cells, all dead cells, and dead tumor cells, obtained by dye co-localization.

FIGS. 27A-27C show spatial cell clustering of CD8 cells. FIG. 27A shows clusters of cells detected using the DBSCAN algorithm. FIGS. 27B & 27C show observed and expected G functions.

FIGS. 28A-28B show immune-immune cell clustering. FIG. 28A is a maximum intensity projection of the CD8⁻ immune cells and CD8⁺ immune cells. FIG. 28B is a pair-correlation analysis of FIG. 28A.

FIG. 29 shows immune-tumor cell engagement.

FIG. 30 is a line graph showing the percent change in number of tumor cells over time for axitinib+pembrolizumab-treated and untreated cells.

FIG. 31 is a line graph showing the number of CD8+ cells that invaded a tumor-cell-containing hydrogel over time for Keytruda-treated and CD3-activated immune cells.

FIG. 32 is a line graph showing the percent change in live tumor cells over time for Keytruda-treated and CD3-activated immune cells.

FIG. 33 is a line graph showing average migration speed of CD8+ cells over time for Keytruda-treated and CD3-activated immune cells.

FIG. 34 is a line graph showing the percent change in tumor-cell-containing hydrogel dome volume over time for Keytruda-treated and CD3-activated immune cells.

FIG. 35 is a line graph showing the percentage of CD8+ cells in clusters over time for Keytruda-treated and CD3-activated immune cells.

FIG. 36 is a line graph showing the number of CD8+/CD8-cells clustered together over time for Keytruda-treated and CD3-activated immune cells.

FIG. 37 is a line graph showing tumor cell viability over time following neutrophil cell therapy.

FIG. 38 is a line graph showing immune cell infiltration over time for various Keytruda and cell therapy treatments.

FIG. 39 is a line graph showing the percent change in tumor cells over time for four test agent combinations and two controls.

FIG. 40 is a line graph showing metabolic activity (left axis) and cell viability (right axis) over time for a hydrogel containing tumor cells.

### DETAILED DESCRIPTION

This disclosure relates to methods of forming three-dimensional (3D) immune-tumor cell cultures from tumor and matched whole blood samples such that the culture recapitulates an in vivo tumor-immune environment. Potential therapeutic agents may be tested in the 3D cultures. The disclosure also includes methods of imaging and analyzing features of and interactions in the 3D culture at a single-cell and single-event level within the context of the entire 3D culture and over time. The disclosed imaging and analysis may be performed by a computer vision pipeline that helps evaluate the effects of the tested agents on the 3D cultures.

Unless defined otherwise below, technical and scientific terms used herein have the same meaning as commonly understood by one of ordinary skill in the art to which this invention pertains. In the case of conflict, the present specification, including definitions, will control. For the purposes of the present invention, the following terms are defined for clarity.

As used herein, “subject” or “patient” means a human or other mammal. Non-human subjects or patients may include, but are not limited to, various mammals such as domestic pets and/or livestock. A subject may be considered in need of treatment. The disclosed methods may be effective to screen healthy subjects or those diagnosed with cancer.

As used herein, “immune” cells refers to peripheral blood mononuclear cells (PMBCs) and any subpopulation thereof, including monocytes, dendritic cells, and lymphocytes, such as B cells, T cells, and natural killer (NK) cells. Subpopulations may also be based on which cluster of differentiation (CD) cell surface molecule(s) the cells express, such as CD8, CD56, and CD14.

The singular terms “a,” “an,” and “the” include plural referents unless context clearly indicates otherwise. Similarly, the word “or” is intended to include “and” unless the context clearly indicates otherwise. The term “comprises” means “includes.” Also, “comprising A or B” means including A or B, or A and B, unless the context clearly indicates otherwise. Although methods and materials similar or equivalent to those described herein may be used in the practice or testing of this disclosure, suitable methods and materials are described below. In addition, the materials, methods, and examples are illustrative only and not intended to be limiting.

### Three-Dimensional Co-Culture Preparation

Three-dimensional co-cultures of the present disclosure include those formed from cells derived from a tissue sample from a subject and a whole blood sample from the same subject. The tissue sample may be from a tumor, such as a cancerous tumor, or from an area adjacent or proximate to the tumor, or from healthy tissue elsewhere in the body.

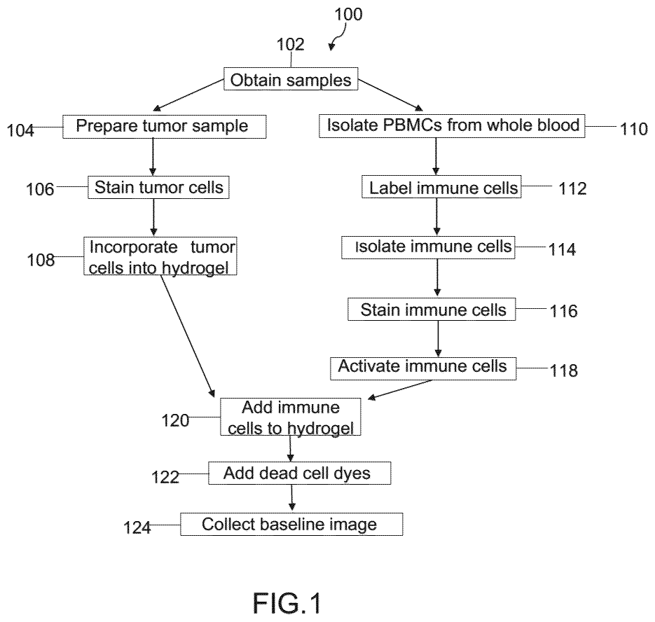

FIG. 1 is a flow chart of a method of according to at least one embodiment of the present disclosure. Method 100 may be a 3D cell culture preparation method. The method 100 may be performed within about 24 hours. Compared to known methods, the presently disclosed method 100 may be performed in less time, which may help the cultured cells maintain their original functionality. Cells prepared and commenced to culture quickly according to the disclosed method 100 may behave more like they do in their native environment than if they had been prepared more slowly or in a different form, such as an organoid.

At block 102, “obtain samples” may be performed. In some embodiments, the samples are from a patient diagnosed with or suspected of having cancer. The samples may include a tissue sample and a blood sample from the same patient.

The tissue sample may be at least a portion of a tumor or healthy tissue, which may be proximate or adjacent the tumor or from elsewhere in the body. For convenience, the term “tumor” is regularly used in the present disclosure, but the sample need not be from cancerous or otherwise diseased tissue, and it should be understood that healthy tissue and its derivative cells could be substituted for “tumor” cells in the cultures and methods disclosed herein. The tissue sample may be obtained from a tumor of any origin, including the breast, kidney, liver, brain, ovary, pancreas, lung, colon, bladder, or stomach. The tumor sample may have been obtained from a surgical resection, core needle biopsy, fine needle aspirate, or the like.

In some embodiments, the samples are fresh. Fresh tumor samples may be received in a tissue transport medium, such as T-Store for tumors. Fresh blood samples may be received in 10 mL K2-EDTA vacutainers. In some embodiments, the samples are frozen. Frozen tumor samples, which may be intact tissue or dissociated cells, may have been frozen in a cryopreservative. Frozen blood samples may include peripheral blood mononuclear cells (PBMCs) that have been isolated from whole blood and frozen in a cryopreservative.

With continued reference to FIG. 1, at block 104, “prepare tumor sample” may be performed. In some embodiments, a fresh or cryopreserved, intact tumor sample is weighed and then processed using a Cytiva VIA Extractor and a digestive enzyme cocktail. In some embodiments, the processing may include temperature changes and/or mechanical digestions. The processing yields a mixed population of dissociated cells, which may include cancer cells, stromal cells, and immune cells (e.g., infiltrating immune cells that were present in a tissue resection or core needle biopsy). The mixed population may be separated into desired cellular subtypes. An example of tumor sample preparation is provided in Example 1.

In some embodiments, a frozen sample of dissociated tumor cells is received at block 102 and block 104 may be omitted.

At block 106, “stain tumor cells” may be performed. Staining tumor cells may permit live cell imaging and tracking. Such imaging and tracking may be performed without affecting cellular function.

In some embodiments, the dissociated tumor cells are stained with at least one dye that stains living cells. The dye may be a cell membrane permeable dye. The dye may stain cells generally or may be specific to a cell subtype or population. The dye may stain a particular region or subpart of a cell, such a lipids, proteins, organelles, cytoplasm, nuclei, or DNA.

In some embodiments, the dye is a fluorescent dye. When two or more dyes are used, each dye may fluoresce at a different wavelength than each other dye. The dye may fluoresce or lose fluorescence in response to a biological process, such as the activation of a cell death pathway, or may not exhibit any change of signal or fluorescence due to a biological process.

The dye may be any living cell dye known in the art. Examples of suitable dyes include BioTracker ATP-Red Live Cell Dye, BioTracker LYSO-TP Live Cell Dye, NucSpot® Live Cell Nuclear Stains, Mitoview, and a membrane-permeable thiol-reactive probe.

An example of tumor cell staining is provided in Example 3.

Referring again to FIG. 1, at block 108, “incorporate tumor cells into hydrogel” may be performed. Incorporating tumor cells into hydrogels results in cell cultures that partially mimic an in vivo tumor environment. Subsequent addition of immune cells, such as that described for block 120, further helps mimic a 3D tumor-immune environment for the accurate study of cancer progression and effectiveness of potential test agents.

In some embodiments, the hydrogel includes a plurality of physiologically relevant components that are configured to mimic core components of human tissue extracellular matrices and/or disease-specific cell niches. Examples of suitable hydrogel components include hyaluronic acid (e.g., 100 kDa molecular weight), extracellular matrix proteins (e.g., collagen I, IV and VI, laminin, fibronectin), proteoglycans, glycoproteins, and growth factors. A hydrogel may also include a basement membrane gel. Any one or more component may be tuned to provide biochemical and mechanical cues that may help the cancer cells to survive, proliferate, and migrate. In some embodiments, one or more of the components may be modified to include or expose an active agents or moiety, such as a vascular endothelial growth factor, oxygen sequestering moiety, or degradation sequence, which may help to accurately model tumor features and behaviors. In some implementations, a functionality, such as oxygen depletion or sequestration, is performed by a media with which the hydrogel is in contact.

In some embodiments, the tumor cells are added to the hydrogel after being stained and/or after being dissociated. In some embodiments, a single population of dissociated cells is added to a hydrogel. In some embodiments, multiple cell types are added to a hydrogel.

In some embodiments, culturing of the tumor-cell containing hydrogel is commenced within one hour of staining the isolated tumor cells 106. Quickly commencing to culture the tumor-cell containing hydrogel may help the tumor cells retain their original patient biology, including by maintaining cell functionality and gene expression profile.

In some implementations, the mixture or suspension of tumor-derived cells and hydrogel is plated on a surface. The surface may be, for example, a cell culture slide, chamber, well, dish, chip, or plate. The mixture may form a dome shape. In one example, about 10,000 differentially stained dissociated tumor cells are encapsulated into a single hydrogel.

In some embodiments, immune cells and/or a test agent may be incorporated into a hydrogel along with the tumor-derived cells. In some embodiments, the test agent includes immune cells (i.e., the immune cells function as a cell therapy). In one example, about 50,000 neutrophil progenitor cells are stained and mixed with about 10,000 differentially stained dissociated tumor cells and encapsulated into a single hydrogel.

In embodiments, the hydrogel is allowed to crosslink. The hydrogel may partially or fully crosslink in less than one hour.

An example of incorporating tumor cells into a hydrogel is provided in Example 3.

At block 110, “isolate PBMCs from whole blood” may be performed. Although shown in parallel to block 104 in FIG. 1, block 110 may be performed up to 24, up to 48, or up to 72 hours prior to block 104 and/or other aspects of tumor processing (e.g., blocks 106 and 108). Alternatively, block 110 may be performed within 24 hours after one or more of blocks 104-108. In some embodiments, the PMBCs are isolated from matched blood. Using matched patient blood may help recreate an immune environment for the tumor cells that accurately replicates a patient's in vivo immune-tumor environment.

In some implementations, isolation includes a series of density-centrifugation steps in a density gradient solvent. Ficoll may be used to separate the whole blood.

In some embodiments, the extracted PBMCs include a population of multiple cell types including monocytes, dendritic cells, and lymphocytes, including B cells, T cells, and natural killer (NK) cells. Flow cytometry may be used to characterize the cell population composition, ratios, and activation status.

In some embodiments, isolated immune cells are received at block 102 and block 110 may be omitted.

An example of isolating PBMCs from whole blood is provided in Example 4.

At block 112, “label immune cells” may be performed. Labeling may help separate subpopulations of immune cells, such as for analysis of subset function or performance. Subpopulations may include CD8⁺, CD8⁻, CD56⁺, CD56⁻, CD14⁺, CD14⁻, and NK cells. In some embodiments, the label includes a magnetic label, such as a magnetic bead.

An example of labeling immune cells is provided in Example 5.

With continued reference to FIG. 1, at block 114, “isolate immune cells” may be performed. The isolation may be of one or more labeled subsets prepared via block 112. In some embodiments, when magnetic labels have been applied to a cell subpopulation, the labeled subpopulation (positive fraction) may be isolated by magnetic activated cell sorting (MACS). The unlabeled subpopulation or subpopulations (negative fraction) may also be retained for subsequent processing. In one example, the positive/negative fractions are CD8⁺/CD8 cells. In one example, the positive/negative fractions are CD56⁺/CD56 cells. In one example, the positive/negative fractions are CD14⁺/CD14 cells.

An example of isolating immune cells is provided in Example 5.

At block 116, “stain immune cells” may be performed. The staining may be of one or more fractions isolated via block 114. Each fraction may be stained with a dye having a different color. Any suitable dye in the art may be used. Examples of suitable dyes include membrane permeable amine- or thiol-reactive fluorescent probes.

In some embodiments, the stained fractions are recombined to form a culture of differentially stained PBMCs. In some embodiments, the fractions are not recombined.

An example of staining immune cells is provided in Example 6.

At block 118, “activate immune cells” may optionally be performed. When performed, activation may allow T cells to rapidly expand and/or to mobilize. Activation may increase T cell function, including targeting and killing tumor cells, compared to no activation.

In some embodiments, immune cells are activated by exposure to a T-cell activating agent, a lipopolysaccharide, a cytokine (e.g., interleukin 2), or a colony stimulating factor. Examples of activating agents include an anti-CD3 antibody, an anti-CD28 antibody, other CD3 and/or CD28 agonists.

In one example, activation includes briefly (<12 hours) culturing isolated and stained PBMCs in a vessel pre-coated with a specific anti-CD3 monoclonal antibody, with or without interleukin-2.

An example of activating immune cells is provided in Example 7.

Referring again to FIG. 1, at block 120, “add immune cells to hydrogel” may be performed. The immune cells may be some or all of those prepared as described for blocks 102 and 110-118. The hydrogel may be prepared with tumor cells as described for blocks 102-108. Adding immune cells to the tumor cell-containing hydrogel may form a 3D immune-tumor cell culture.

In some embodiments, the immune cells are added to the tumor cell-containing hydrogel within 24 hours of commencing to culture the tumor cell-containing hydrogel. Adding immune cells soon after commencing the tumor-hydrogel culture may help the resulting 3D culture retain the original patient biology, including by maintaining cell functionality and gene expression profile.

In some embodiments, the immune cells are added in a solid or liquid medium to the hydrogel, such as around the hydrogel. The solid medium may be a hydrogel, and the second hydrogel (containing immune cells) may be added on top of or around the first hydrogel (containing tumor cells). The liquid medium may be a primary cell culture medium. In some embodiments, the immune cells are added in a suspension to an exposed surface of the hydrogel. The exposed surface may be the top or convex portion of the hydrogel.

The tumor cell to immune cell ratio may be from about 1 to 1 to about 1 to 100, about 1 to 5 to about 1 to 50, or about 1 to 5 to about 1 to 10. In some embodiments, block 120 is not performed, and the resulting culture without immune cells may serve as a control for a different culture that includes immune cells.

Additionally or alternatively, immune cells may be added to a tumor-cell containing hydrogel one or more times after adding a test agent 202, as described below.

An example of adding immune cells to a hydrogel is provided in Example 8.

At block 122, “add dead cell dye” may be performed. Adding at least one dead cell dye may enable the detection of dead cells, such as tumor cells killed by immune cells. In some embodiments, the dye is a membrane-impermeable dye. A membrane-impermeable dye may stain DNA after the cell membrane has ruptured. An example dye is Annexin V, which stains phosphatidylserine and can detect loss of plasma membrane integrity in apoptotic cells. In some embodiments, the dye is a membrane-permeable dye. The one or more dyes may be added to the cell culture supernatant.

An example of adding dead cell dyes is provided in Example 9.

At block 124, “collect baseline image” may be performed. Collecting at least one baseline image may help establish the initial features of a 3D immune-tumor culture, such as the number of each cell type, the XYZ location of each cell, and the viability of each cell.

The images may be collected with live-cell microscopy, which may include one or more of confocal, widefield, lightsheet, and multi-photon microscopy.

An example of baseline imaging is provided in Example 10.

### Testing Agents in Three-Dimensional Co-Culture

The 3D immune-tumor cell cultures disclosed herein, such as those produced by method 100, may be used as tools for evaluating the performance of an agent, such as potential chemotherapeutic or immunotherapy agent. Testing an agent in a tumor-immune environment that accurately recapitulates an in vivo tumor-immune environment may produce results that translate better to clinical efficacy than results from other testing methods that do not replicate in vivo environments as accurately. The presently disclosed cultures and methods also allow for personalized medical decisions when one or more agents is tested in a cell culture derived from a patient and treatment decisions for the patient are made based on performance of the test agent(s) in the culture. The presently disclosed cultures and methods may also be used for discovering drug targets and/or validating drug candidates.

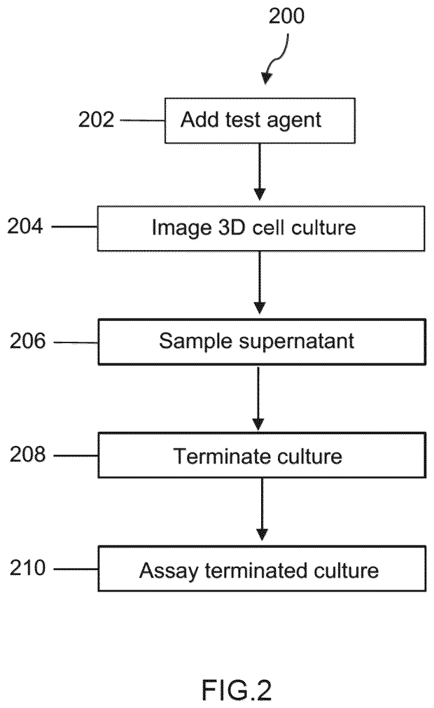

FIG. 2 is a flow chart of a method of according to at least one embodiment of the present disclosure. Method 200 may be a method of testing agents in 3D immune tumor cell cultures. The method 200 may be performed while maintaining a 3D immune-tumor cell culture as an intact 3D immune-tumor cell culture. For example, the method 200 may be performed without mechanically disturbing or disrupting the integrity of the culture, any cell within the culture, or any portion of the hydrogel. The method 200 may be performed without damaging or inactivating the culture or any cell within the culture. As another example, the method 200 may be performed without significantly decreasing or otherwise compromising the viability of the culture or any cell within the culture, except as a result of the effect of the test agent(s). The method 200 may be performed on a single 3D culture rather than utilizing different cultures or portions of cultures for different assays. The method may be performed using a live cell microscope, such as a confocal microscope, as the only required instrument, with the exception of a, for example, pipette used for block 206. The method 200 may be performed without one or more of instrument transfer, reagent transfer, or user intervention after establishing the 3D immune-tumor cell culture.

At block 202, “add test agent” may be performed. The test agent may be selected from one or more of a small molecule, a large molecule, a soluble immunosuppressive-signaling inhibitor, a checkpoint inhibitor, an immune activator, a virus, a bacterium, a gene therapy, and a cell therapy. Examples of a large molecule include an antibody, protein, peptide, drug conjugate, or nucleic acid, such as siRNA. Examples of an immune checkpoint inhibitor include pembrolizumab, ipilimumab, nivolumab, and atezolizumab. Examples of cell therapy include lymphocyte-based therapy and myeloid-based therapy. Such therapies include lymphocytes or myeloid cells that are unaltered, refined (e.g., contain a selection of specific cell subpopulations with anti-tumor activity), or altered (e.g., include the addition of CARs). Examples of lymphocyte-based therapy include a T-cell receptor therapy and a chimeric antigen receptor (CAR) T-cell therapy. In some embodiments, the cell therapies are allogenic. In some embodiments, the immune cells prepared as described for block 110 et. seq. (i.e., from a patient-matched blood sample) are themselves a test agent.

In some embodiments, no test agent is added to a tumor-immune culture and the culture serves as a negative control for another culture to which a test agent has been added.

Additionally or alternatively, a test agent may be added to stained immune cells, such as at block 116 and/or 118 of method 100. Additionally or alternatively, a test agent may be added concurrently with adding immune cells to a hydrogel, such as at block 120.

An example of adding a test is provided in Example 11.

At block 204, “image 3D cell culture” may be performed. Images may be collected over time to evaluate cell metrics over time, as described in more detail below for method 300.

The images may be collected with live-cell microscopy, which may include one or more of confocal, widefield, lightsheet, and multi-photon microscopy. In one example, 3D immune-tumor cultures are imaged by 3D confocal microscopy at 60-minute intervals for 16 hours. In one example, 3D immune-tumor cultures are imaged by 3D confocal microscopy each day for 4 days.

An example of adding a test agent is provided in Example 12.

With continued reference to FIG. 2, at block 206, “sample supernatant” from a 3D co-culture may be performed. The supernatant may be evaluated for the presence of soluble factors such as cytokines, chemokines, growth factors, and other immuno-regulatory factors. Soluble factor levels may be measured by any method known in the art, such as ELISA or Luminex. In some embodiments, supernatant samples are collected regularly, such as each day a 3D culture is being maintained. Additionally or alternatively, the supernatant may be sampled when or after the culture is terminated, such as described for optional block 210.

An example of sampling a supernatant is provided in Example 13.

At block 208, “terminate culture” may be performed. The 3D immune-tumor cell cultures disclosed herein may be maintained for a desired length of time, such as up to 14 days, up to 10 days, up to 6 days, or about 3 to 12 days or about 4 to 7 days. In some embodiments, the culture is terminated by stopping the 3D culture and disposing of it. In some embodiments, the 3D culture is terminated by fixing it. In some embodiments, the 3D culture is terminated by digesting the hydrogel and extracting some or all of the remaining material, such as cells, nucleic acids, or proteins.

At optional block 210, “assay terminated culture” may be performed. The terminated culture may be subject to one or more evaluative methods to further analyze features of the culture and its response to a test agent. Post-termination methods include measuring metabolic activity, determining cell viability, and measuring RNA expression profiles and levels. Measuring metabolic activity may be performed using a cell viability substrate and 1×NanoLuc Enzyme and measuring the resulting luminescence. Determining cell viability may include a live/dead assay and/or imaging fluorescence of the cells of the culture with confocal microscopy.

In some implementations, assaying a terminated culture 210, such as sampling a supernatant and analyzing the sample with, for example, an ELISA or Luminex assay, may provide a readout of exhaustion of immune cells at the molecular level. Any combination of samples or analyses from block 210 and/or 206 may provide a readout of exhaustion of immune cells at the molecular level.

Examples of post-termination analyses are provided in Example 14.

### Computer Vision Pipeline

An automated computer vision (CV) pipeline may be used to detect individual cells from a stack of 3D images. In some embodiments, the images are acquired using a confocal fluorescence microscope. Each individual fluorescent dye may be acquired in a separate channel. The CV pipeline may be capable of processing single images acquired at one or more time points, as well as time-lapse images.

In some embodiments, a cell culture disclosed herein is imaged daily over the course of many days and the images are processed to measure several metrics, including immune cell counts, tumor cell counts, tumor cell viability, immune cell infiltration, and the XYZ-time co-localization between any two or more cells, cell types, and/or dyes. Changes in any or all of these features may be monitored over time.

In some embodiments, time-lapse microscopy is used to image the same culture at a desired interval, such as every few minutes or every hour, to enable the accurate XYZ-time tracking of tumor cells, immune cells, and other cells of interest. These measurements may be used to calculate the speed and distance migrated of any individual cell or a population of cells.

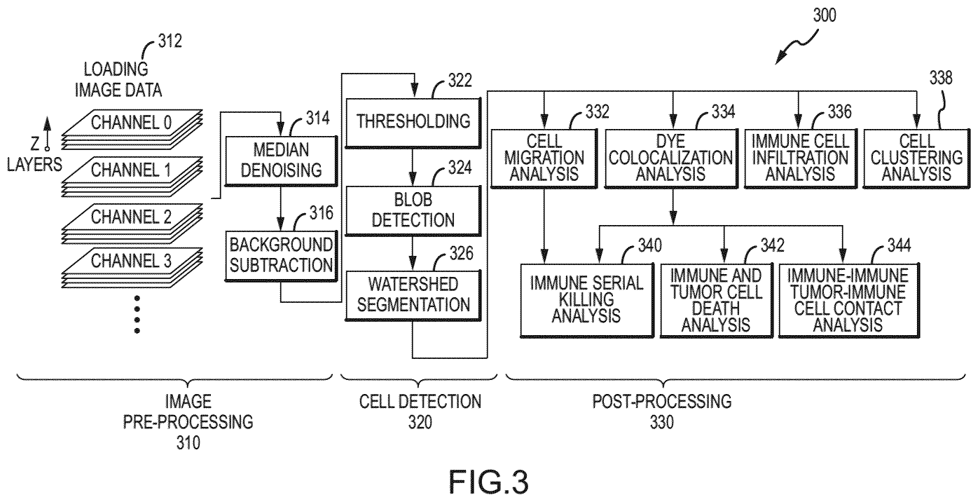

FIG. 3 is a flow chart of a method of according to at least one embodiment of the present disclosure. Method 300 may be a CV detection pipeline. The method 300 may be implemented to track and study features of a cell culture, such as a culture prepared by the methods disclosed herein. The method 300 may be performed while maintaining a 3D immune-tumor cell culture as an intact 3D immune-tumor cell culture. For example, the method 300 may be performed without mechanically disturbing or disrupting the integrity of the culture, any cell within the culture, or any portion of the hydrogel. The method 300 may be performed without damaging or inactivating the culture or any cell within the culture. As another example, the method 300 may be performed without decreasing or otherwise compromising the viability of the culture or any cell within the culture. The method 300 may be performed on a single 3D culture rather than utilizing different cultures or portions of cultures for different assays. The method may be performed using a live cell microscope, such as a confocal microscope, as the only required instrument. The method 300 may be performed without one or more of instrument transfer, reagent transfer, or user intervention after establishing the 3D immune-tumor cell culture. The data gathered in method 300 may be exclusively phenotypic data.

Performance of the method 300 may reveal at least one difference between an effect of a test agent and a control agent on the disclosed 3D immune-tissue cell cultures. Potential effects are described in more detail below and may include one or more of number of the immune cells, death of the immune cells, interactions between immune cells, immune cell infiltration of the cell-containing hydrogel, immune cell engagement of the tissue-derived cells, immune cell killing of the tissue-derived cells, immune cell serial killing of the tissue-derived cells, death of the tissue-derived cells, and exhaustion of the immune cells. See blocks 132-144, supra.

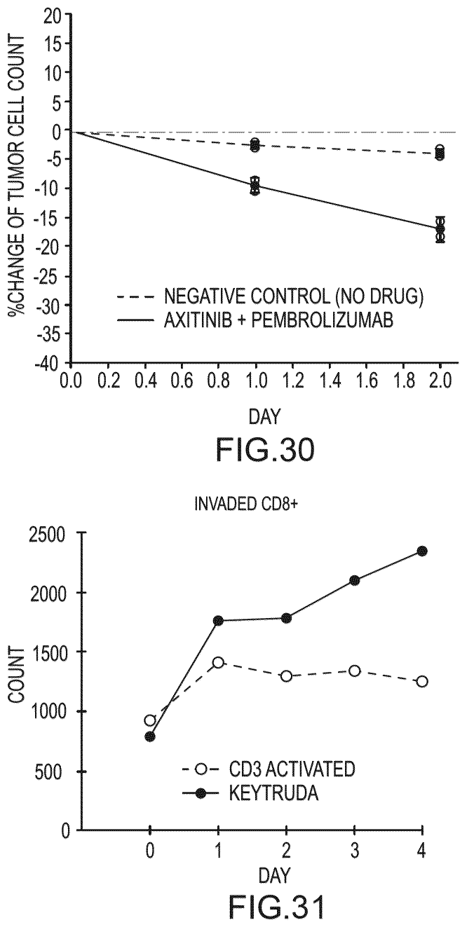

The difference may be quantifiable soon after adding the test agent, such as within about 24 to about 48 hours of, or about 48 hours after, adding the test agent. In some implementations, the difference may be a statistically significant difference. Measurable, significant, clinically relevant, and/or therapeutically relevant results from a test agent may be available via the presently disclosed methods sooner than via known methods. Known methods may be methods of forming a 3D immune-tumor cell culture in which the culture is formed more than 24 hours after commencing to culture the tumor cells. Known methods may be animal models of cancer comprising immunodeficient mice reconstituted with a human immune system. One example of such an animal model is of triple-negative breast cancer (TNBC) and includes immunodeficient reconstituted BRGS-HIS mice engrafted with TNBC cell line xenografts (CDXs). (“Immunotherapy Efficacy Assessment and Metastases Studies: This Model is Highly Permissive to Engraftment,” November 2021, Genoway, available at https://www.genoway.com/commentaries/tumor-grafts2.htm). In the animal model, nivolumab (an anti-PD-1 immunotherapy) did not show a significant effect relative to the vehicle control on tumor volume until 10 days after treatment. In contrast, a difference in tumor (dome) volume between an immune-tumor culture treated with an anti-PD-1 immunotherapy (i.e., Keytruda/pembrolizumab) and a control culture is measurable, and noticeable, and may be statistically significantly different, by one day after adding the treatment. (See Example 17 and FIG. 34.)

The method 300 may include image pre-processing 310, cell detection 320, and post-processing 330.

The image pre-processing 310 may be used to take stacks of images through a culture volume (z-stack) and prepare them for subsequent cell detection 320.

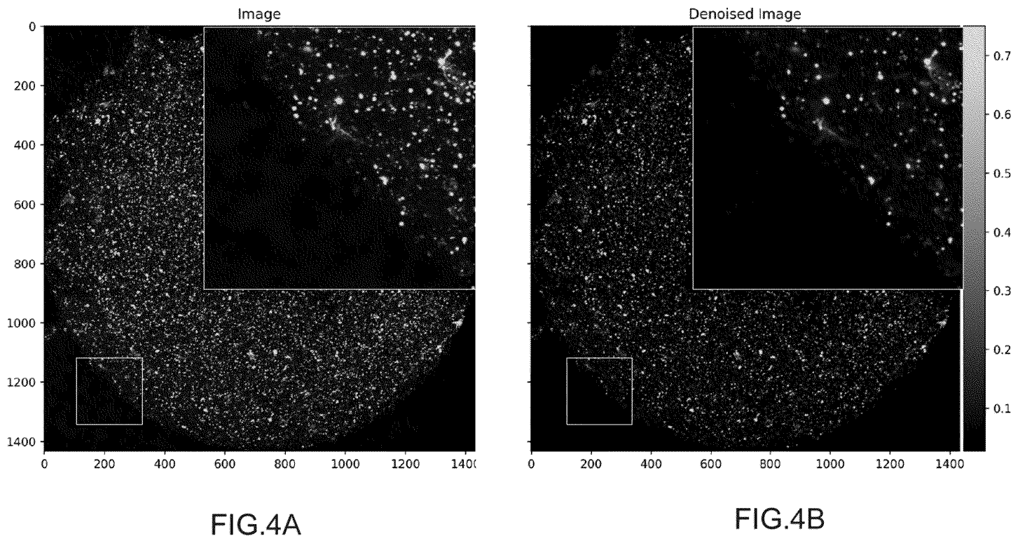

In image pre-processing 310, “loading image data” 312, “median denoising” 314, and “background subtraction” 316 may be performed. Loading image data 312 may include reading 3D multi-channel images and converting them to a suitable format for analysis. Median denoising 314 may include a median denoising filter with a 3×3×3 kernel to remove shot noise from the image. FIG. 4 shows an example of a median denoising filter applied to a tumor cell image. The figure shows maximum intensity projections of a tumor cell culture (with tumor cells visualized in yellow), before (FIG. 4A) and after (FIG. 4B) the application of a median denoising filter. A zoomed-in detail of the edge of the culture is shown on the top right of each image. The x-axis and y-axis labels indicate the horizontal and vertical dimensions of the image and correspond to the columns and rows of the 2D image being displayed. The color map on the far right represents the mapping of image intensity values to colors. The image intensities were rescaled to be in the 0-1 range and the colormap was adjusted to the 0-0.75 range to enhance dim features.

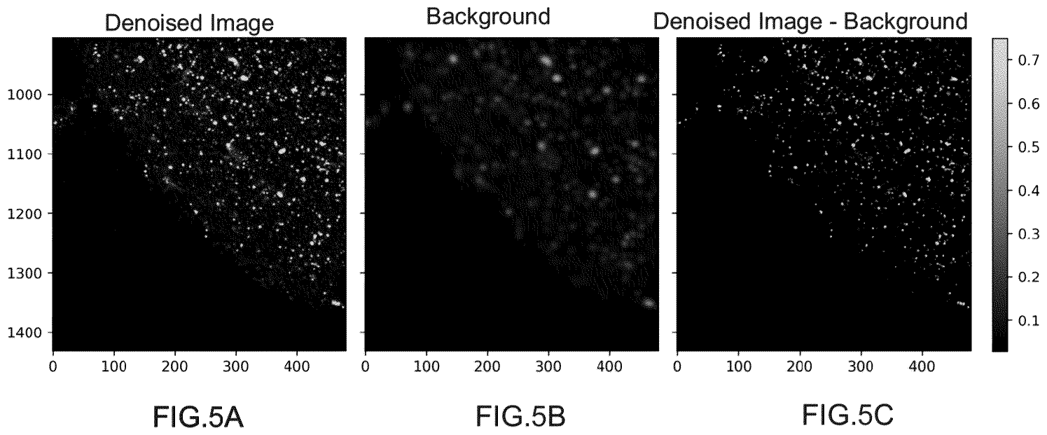

Background subtraction 316 may be performed by a background subtraction filter. Background subtraction 316 may include removing background auto-fluorescence, removing the smoothly varying background, preserving smaller, finer details (e.g., foreground objects), and/or removing other imaging artifacts. In some embodiments, background subtraction 316 may include one or more of a rolling ball algorithm, a top-hat algorithm, a Gaussian smoothing background subtraction algorithm, and a wavelet-based background subtraction algorithm. For each algorithm, the choice of the kernel size may be based on the size of the largest feature that is desired to be retained. Given the average size range of the cell type that is represented in a particular channel (usually between about 5 and about 20 μm in diameter), the kernel size can be calculated from the image voxel size in μm. FIG. 5 shows an example of a wavelet-based background subtraction algorithm applied to the denoised tumor cell image shown in FIG. 4. FIG. 5 shows zoomed-in maximum intensity projections of the edge of a tumor cell culture (with tumor cells visualized in yellow). FIG. 5A shows the denoised image; FIG. 5B shows the background image; and FIG. 5C shows the image after background subtraction. The x-axis and y-axis labels indicate the horizontal and vertical dimensions of the image and correspond to the columns and rows of the 2D image being displayed. The color map on the far right represents the mapping of image intensity values to colors. The image intensities were rescaled to be in the 0-1 range and the colormap was adjusted to the 0-0.75 range to enhance dim features.

Referring again to FIG. 3, in cell detection 320, segmentation algorithms may be used to separate individual cells from the background and/or from neighboring cells. In some embodiments, “thresholding” 322, “blob detection” 324, and/or “watershed segmentation” 326 may be performed.

Image thresholding 322 may separate background objects from foreground objects. In some embodiments, thresholding 322 is performed locally and different thresholds for different regions of an image are set based on local pixel/voxel characteristics. In some embodiments, image thresholding is performed globally and a single threshold is applied to an entire image.

In some embodiments, the thresholding 322 is performed globally and an algorithm is used to find the intensity values that best separate the background and foreground objects. In some embodiments, a measure of the background mean and standard deviation from a region of the image that does not contain any foreground objects is used. In some embodiments, a global threshold is determined using a histogram-based method suitable for unimodal histograms, including Rosin's triangle thresholding and T point thresholding methods.

Blob detection 324 may detect objects that have a rounded shape and a size range compatible with the cell type of interest. In some embodiments, a scale invariant laplacian of Gaussian blob detection method is used. Such method includes the selection of a minimum and a maximum value of sigma, which are used to detect objects of the desired size, and an intensity threshold, which is used to filter fluorescent objects that are too dim. The sigma range may be set to the average size range of the cell type of interest, while the threshold may be set to the global threshold determined by thresholding 322. Application of such a filter may produce a list of the XYZ coordinates of the detected objects' centroids and their associated radii.

Watershed segmentation 326 may include an algorithm to separate objects into individual labels. Watershed segmentation 326 may include calculating features of interest, such as cell centroid XYZ coordinates, cell sizes (volume and/or its equivalent pixel diameter), and mean and/or maximum fluorescent intensities. In some implementations, objects' centroids from blob detection 324 and binary images obtained from thresholding 322 are used in watershed segmentation 326 to refine the boundaries of the detected cells and separate overlapping cells. Such application may produce an annotated image where each detected object is described by a label identifier.

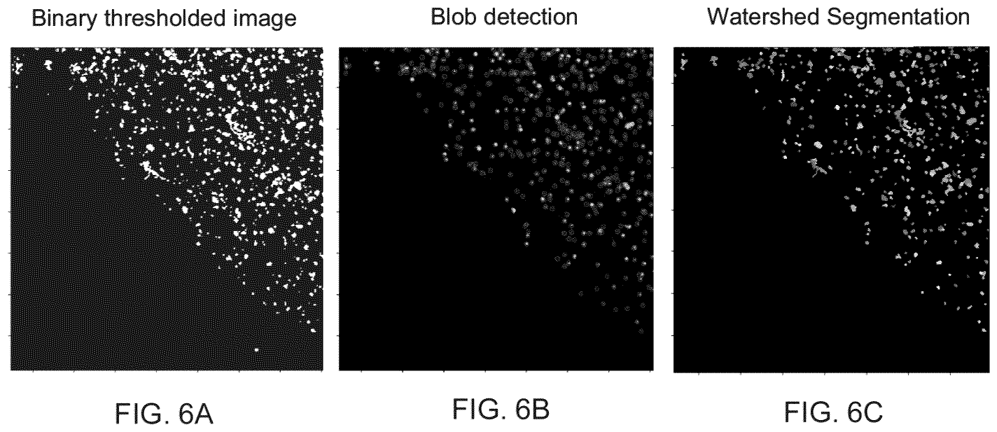

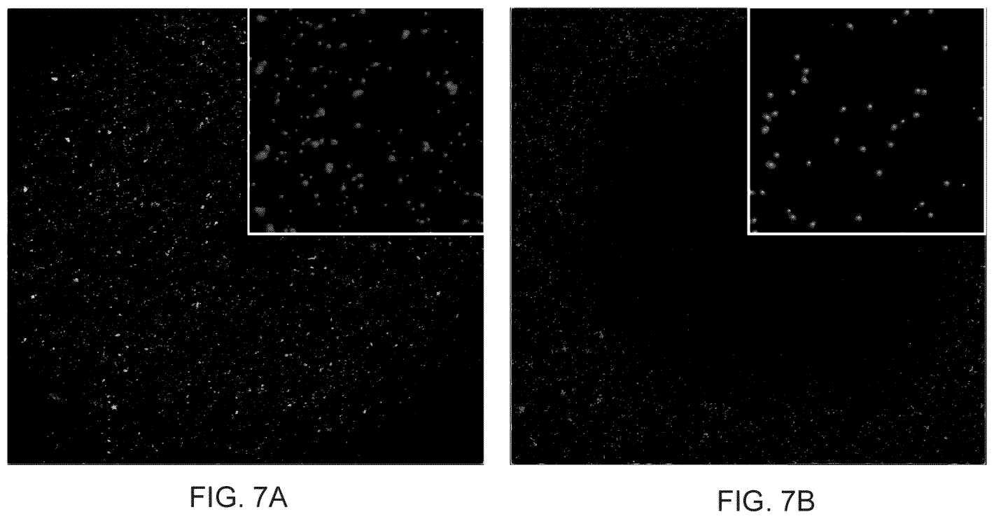

FIG. 6 shows an example in which global thresholding 322, blob detection 324, and watershed segmentation 326 have been applied to a tumor cell image. The figure shows zoomed-in maximum intensity projections of the edge of a tumor culture. FIG. 6A shows a binary image obtained after thresholding 322 the pre-processed image; FIG. 6B shows blob-like objects (red circles) detected using a blob detection 324 algorithm; and FIG. 6C shows the segmented cells (each color corresponds to an individual cell) obtained from watershed segmentation 326.

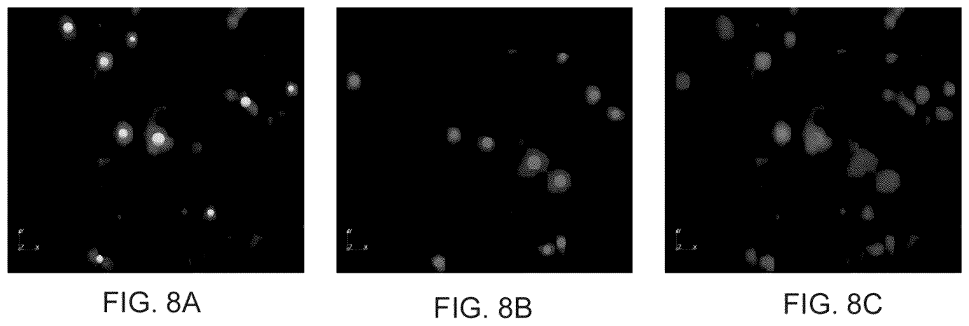

FIG. 7 shows examples of cell detection. Maximum intensity projections of tumor-immune culture image are shown with their corresponding cell detections represented as spheres. Cell detections were overlaid on fluorescent images for two channels. FIG. 7A shows the tumor cells visualized in yellow and the cell detections visualized as red spheres; and FIG. 7B shows CD8⁻ immune cells visualized in red and the cell detections visualized as white spheres.

Each single cell may be associated with its corresponding XYZ location in the collected image and physical position in the microscope stage, time index (in case of multi-time point imaging), fluorescent intensity, and channel or dye information. A suitable data structure (table or dataframe) may be used to store the detected object information for further analysis.

With continued reference to FIG. 3, in post-processing 330, cells detected in cell detection 320 are further analyzed to produce one or more summary metrics. Metrics may include tumor cell counts, immune cell counts, tumor cell death, tumor cell viability, immune cell death, immune cell viability, immune infiltration into a cell-containing hydrogel, interactions between immune cells, clustering of immune cells, immune cell engagement of tumor cells, immune cell killing or serial killing of tumor cells, and exhaustion of immune cells.

In post-processing 330, “cell migration analysis” 332, “dye co-localization analysis” 334, “immune cell infiltration analysis” 336, “cell clustering analysis” 338, “immune serial killing analysis” 340, “immune and tumor cell death analysis” 342, and “immune-immune and tumor-immune cell contact analysis” 344 may be performed.

Dye co-localization 334 may include the analysis of objects labeled with different dyes. The objects may be cells, cell organelles, or other cell structures. The dyes may be fluorescent dyes. Dye co-localization 334 may include studying the degree of spatial overlap or co-occurrence between distinct dye-labeled components within a sample. The analysis may help determine whether different cellular structures or biomolecules of interest are co-localized within the same cells or regions, which may indicate potential interactions or functional relationships.

In some embodiments, dye co-localization 334 employs a distance-based co-localization method based on K-nearest neighbors (KNNs). Such a method may assess the spatial relationship between two sets of cell centroids labeled with different fluorescent dyes. Such a method may measure the number of co-localized spots by analyzing the distances between points from one set to their nearest neighbors in the other set.

In some embodiments, dye co-localization 334 includes first obtaining two sets of points from different color channels (e.g., channel 1 and channel 2), such points representing the centroids or spatial coordinates of cells labeled with distinct fluorescent dyes. Then K-Nearest Neighbors are calculated whereby, for each point in set A (from channel 1), its K-nearest neighbors are found in set B (from channel 2) based on Euclidean distance or other distance metrics. At a minimum, one (K=1) nearest neighbor is used in the search. Next, for each point in set A, the average distance to its K-nearest neighbors in set B is computed. Then, a distance threshold or criterion based on experimental considerations or the specific biological context is determined. Points with average distances below the threshold may indicate co-localization, which may suggest that cells (or cellular components) from set A are closely associated with cells (or cellular components) from set B.

In some implementations, dye co-localization 334 is performed between tumor cells and spots obtained from a dead cell dye that fluorescently stains tumor cells to calculate the number of dead or dying tumor cells. The distance threshold may be set to, for example, 5 μm because the cell components of a dying or dead cell stained with live and dead cell dyes are expected to be overlapping and thus the centroids very close.

In some implementations, dye co-localization 334 is performed between immune cells and dead cell dyes to quantify immune cell death. The distance threshold may be set to, for example, 5 μm because the cell components of a dying or dead cell stained with live and dead cell dyes are expected to be overlapping and thus the centroids very close.

In some implementations, dye co-localization 334 is performed between tumor cells and immune cells to quantify the immune-tumor engagement. The distance threshold may be set to, for example, the sum of both cell radii plus a fixed tolerance distance, as two cells in contact with each other do not physically overlap.

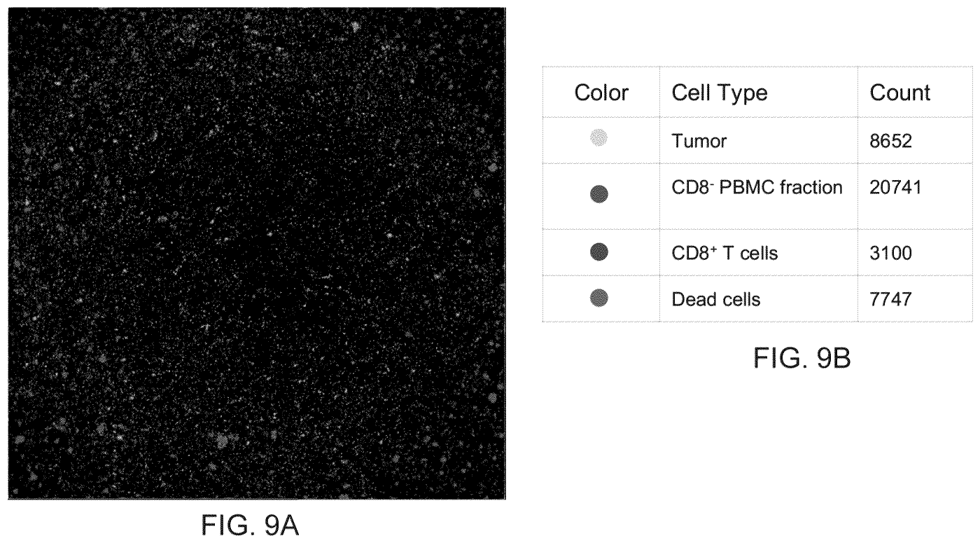

FIG. 8 shows example images of two dyes co-localizing in the same cell. FIG. 8A is of tumor cells and corresponding detections (in yellow); FIG. 8B is of dead cells and corresponding detections (in green); and FIG. 8C shows the two dyes co-localize in two cells (in red).

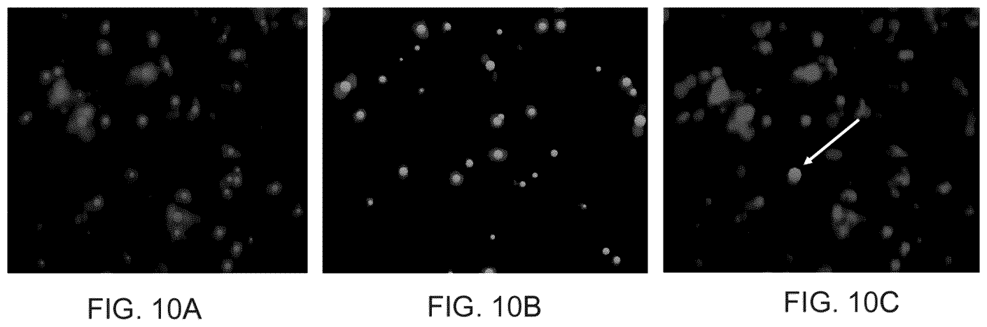

Following the foregoing cell detection 320, each identified cell is represented as an individual object in an image. Each group of identified cells and derived objects (co-localized spots) is counted to determine the total number of cells in the image. FIGS. 9A & B show an example of cell counts obtained from an immune-tumor culture. FIG. 9A is a maximum intensity projection of a tumor-immune culture in which tumor cells, CD8⁺, and CD8⁻ cells are visualized by yellow, blue, and red, respectively, and green areas indicate cell death. FIG. 9B indicates cell counts from the associated channels.

With continued reference to FIG. 3, cell migration analysis 332 may include sequential immune cell detections from time-course image data.

In some embodiments, cell migration analysis 332 employs linear assignment problem-based cell tracking, which may make temporal associations of cells in 3D time-course data. Such tracking may use features extracted in cell detection 320, such as centroid coordinates (XYZ), cell size, cell shape, and dye or fluorescence intensity, to perform frame-to-frame association. In some implementations, the linear assignment problem is solved using the Hungarian algorithm. The linear assignment problem may assign detected cells in the current frame to existing tracks from the previous frame based on minimizing the total cost of associations. New tracks may be initialized for newly detected cells (e.g., due to proliferation) that cannot be associated with existing tracks in previous frames, and may be terminated for cells that disappear (e.g., due to lysis) from the image or split into multiple cells.

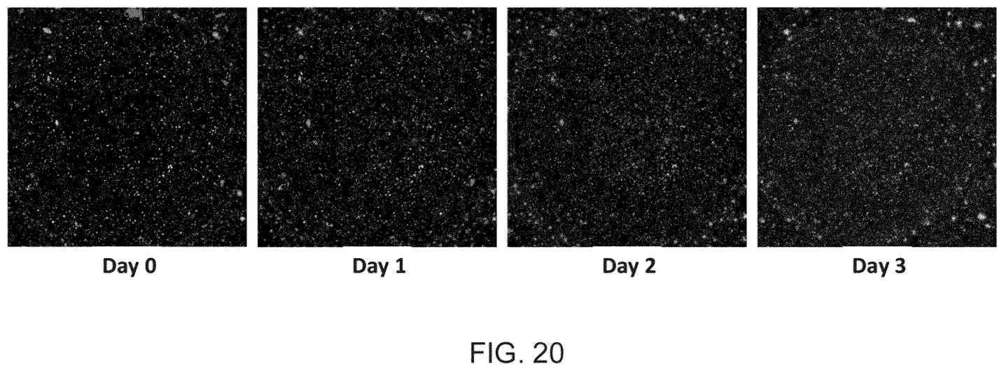

Once cell tracking is completed, summary metrics such as the mean and standard deviation of speed (or velocity) and total migration distance (or displacement) may be calculated. Metrics can be calculated from individual cells, specific cell populations, or certain percentiles (e.g., the top or fastest 5% of migrating cells). An example of each of immune and tumor cell migration analysis 332 is shown in FIGS. 23A-FIG. 23B/. (See Example 15.) The analysis was performed on a one-hour time-lapse with a four-minute interval between each frame. CD8⁺ cells were tracked by solving the linear assignment problem and the average migration speed was calculated.

In some implementations, cell migration analysis 332, such as immune cell migration speed, is used as a proxy for exhaustion of immune cells.

With continued reference to FIG. 3, immune cell infiltration analysis 336 may include counting the number of immune cells that are within the cell-containing hydrogel. A hydrogel region may be identified in advance or in conjunction with the counting.

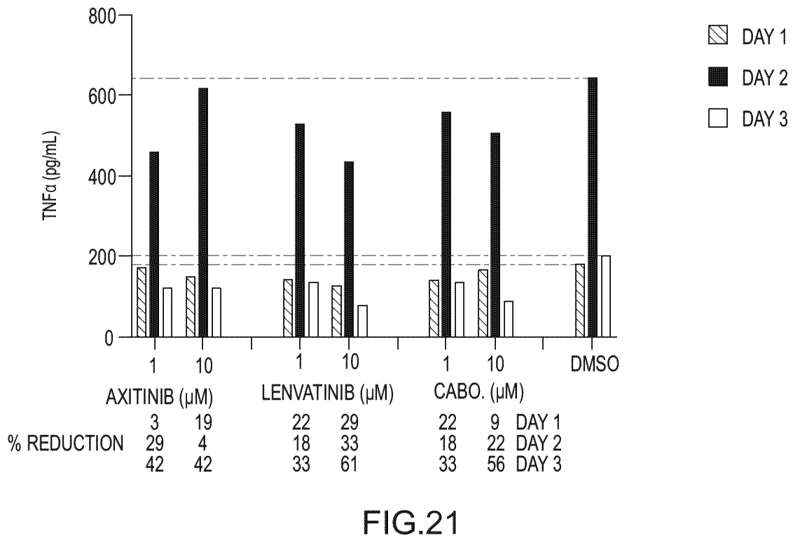

In some embodiments, the cell-containing hydrogel region is formed as a droplet of hydrogel on the bottom of a microplate well. In such cases, the hydrogel boundary is determined during post-processing 330 using the tumor cells' cell centroids: spots belonging to high density regions where many KNNs neighbors are present are retained while sparse and isolated points are removed. Then, the surface enclosing these points is determined using the 3D convex hull algorithm. After the hydrogel surface is determined, the infiltrated immune cells can be filtered, counted, and tracked. According to the culturing methods disclosed herein, immune cells in a culture medium are originally placed on the surface of a tumor-cell-containing hydrogel culture such that identification of immune cells in the hydrogel side of the hydrogel-immune cell interface indicates immune cells that have infiltrated the hydrogel. FIG. 24 of Example 15 is illustrative of immune cell infiltration analysis 336 performed on an immune-tumor culture. The detected tumor cells were used to identify the interface between the hydrogel and culture medium, which was then used to filter the infiltrated CD8⁻ immune cells.

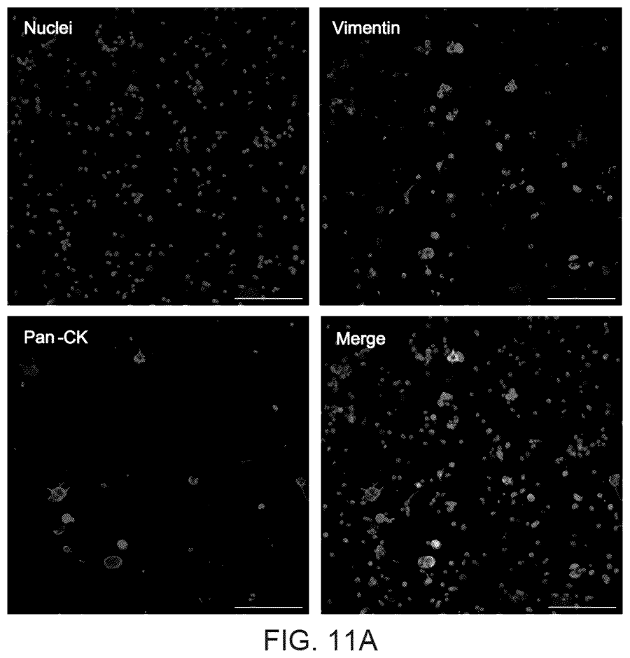

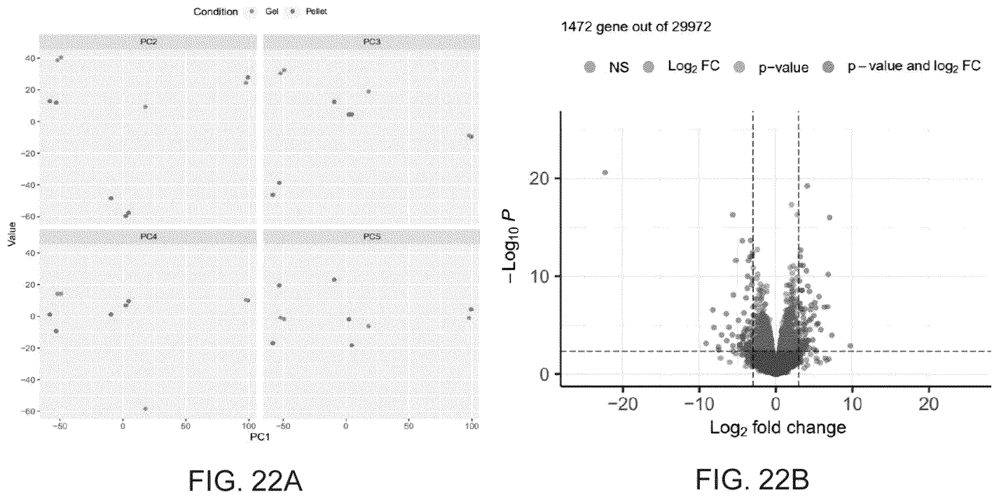

With reference again to FIG. 3, post-processing 330 includes immune and tumor cell death analysis 342. In embodiments, immune cell death may be quantified by measuring the count and/or proportion of dead immune cells. For example, a dye co-localization analysis 334 may be performed between the immune and dead cell dye channels. An example image of immune cell death is shown in FIGS. 10A-C. FIG. 10A shows the CD8⁻ immune cells and corresponding detections (in red). FIG. 10B shows the dead cells and corresponding detections (in green). FIG. 10C shows the two dyes co-localize in one cell (violet sphere indicated by the white arrow). Another example is shown in FIG. 25. (See Example 15.)

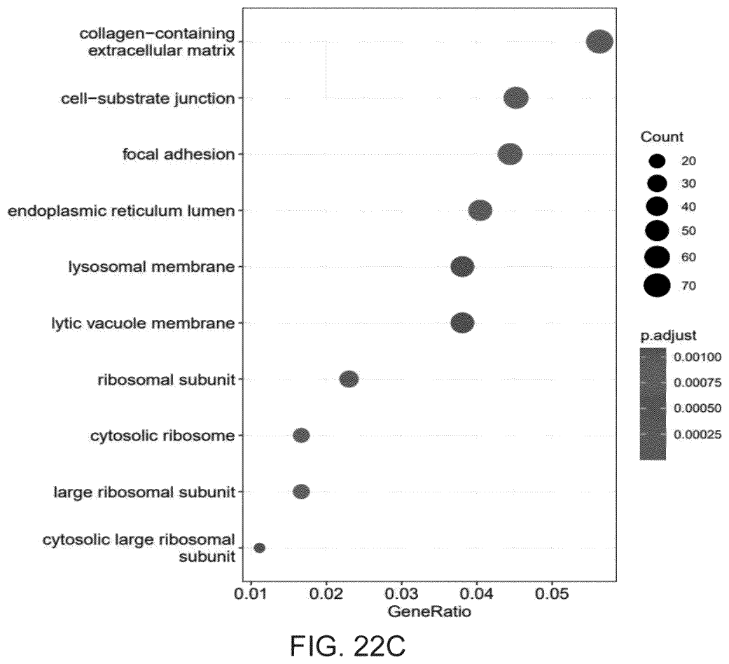

In embodiments, tumor cell death may be quantified by counting the number of tumor cells that co-localize with dead cell dye spots. An example is shown in FIG. 26. (See Example 15.)

With continued reference to FIG. 3, post-processing 330 may include cell clustering analysis 338. In embodiments, spatial statistics may be used to quantitatively assess the spatial organization of immune cells in immune-tumor cell cultures. Identifying spatial clustering patterns may provide information related to an immune response, cell-cell interactions, and the role of immune cells in various diseases or biological processes. For example, the clustering of immune cells may indicate the mounting of an anti-tumor immune response, and/or the formation of a tertiary lymphoid structure or a precursor to a tertiary lymphoid structure. As further examples, immune cell clustering may indicate local areas of cross-presentation between lymphocytes and antigen presenting cells, and/or areas of dynamic cell-cell contact-dependent phenotypic rewiring in response to tumor antigens (e.g., differentiation and proliferation of cytotoxic T lymphocytes).

In embodiments, after cell detection 320, cell coordinates from one or more cell types may be analyzed using univariate methods, such as Ripley's K and G function, Quadrat analysis, spatial autocorrelation, or nearest neighbor analysis. Alternatively or additionally, cell coordinates from one or more cell types may be correlated with other cell types using bivariate methods, such as multivariate Ripley's K and G function, pair correlation analysis, or multivariate nearest neighbor.

Pair correlation analysis, also known as the pair correlation function or radial distribution function, is a statistical method used to analyze the spatial arrangement or distribution of points in a dataset, similar to Ripley's K and G function.

The pair correlation function (PCF) quantifies the probability of finding a neighboring point at a specific distance from a reference point, relative to what would be expected in a random distribution. This analysis helps reveal whether points are clustered, dispersed, or randomly distributed within a given area.

The bivariate pair correlation function is a statistical method used to analyze the spatial relationship between two different types or classes of points in a dataset. It extends the concept of the pair correlation function (PCF) to compare the joint distribution of points from two classes within a common study area. The bivariate pair correlation function provides insights into how the two classes of points interact or influence each other's spatial distribution.

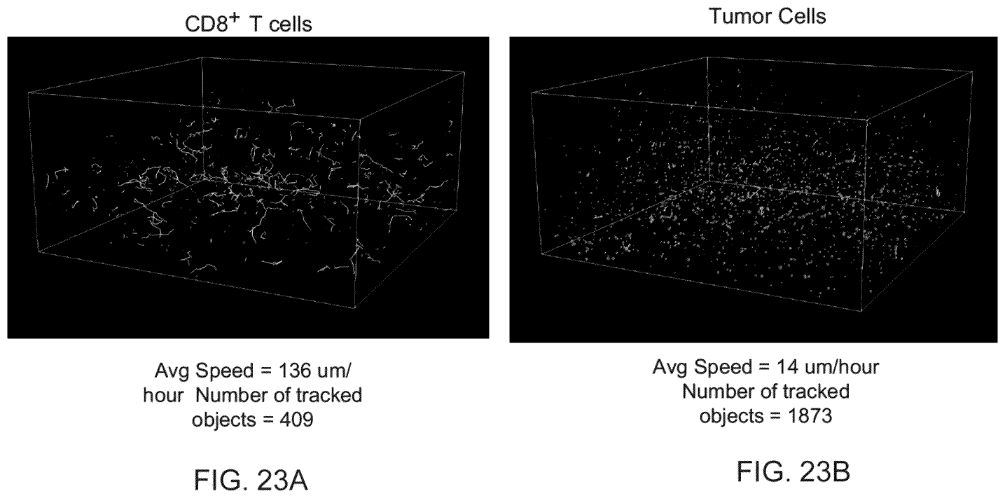

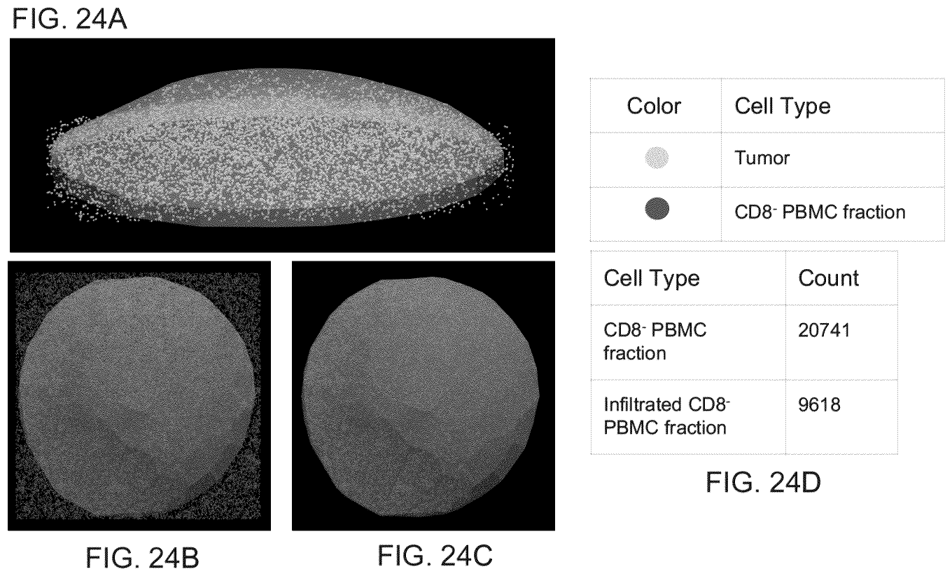

An example of cell clustering analysis 338 using standard pair-correlation analysis is shown in FIGS. 27A-C and an example using bivariate pair-correlation analysis is shown in FIGS. 28A-B. (See Example 15).

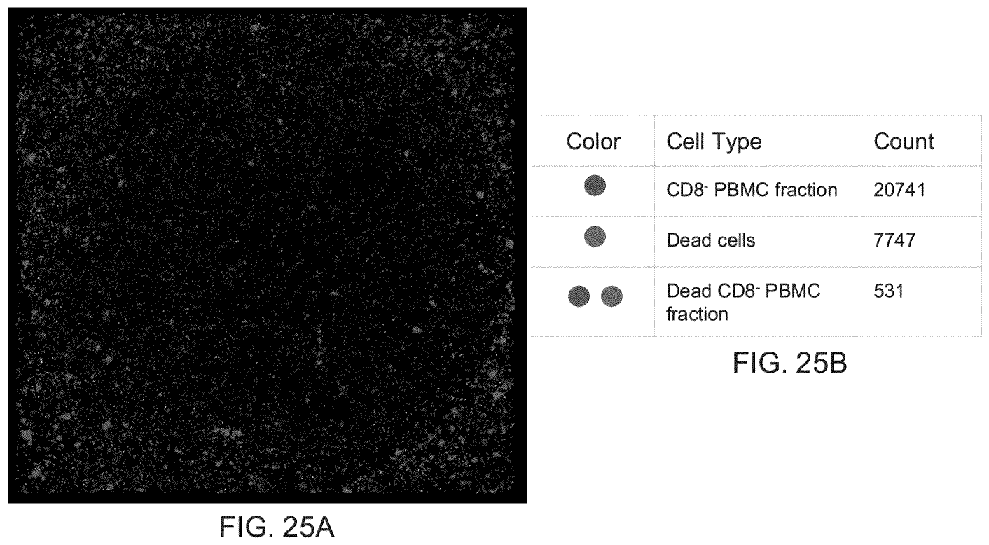

With reference again to FIG. 3, post-processing 330 may include immune-immune and tumor-immune contact analysis 344. The analysis may include immune-tumor engagement. In embodiments, immune-tumor engagement is quantified by counting the number of tumor cells in contact with immune cells. For example, a dye co-localization analysis 334 may be performed between immune cells and tumor cells. The distance threshold may be set to, for example, the sum of both radii of each pair of neighbors, plus a fixed tolerance distance, as two cells in contact with each other do not physically overlap. An example of tumor-immune contact analysis 344 is shown in FIG. 29. (See Example 15).

Immune serial killing analysis 340 may include tracking individual immune cells and counting the number of tumor cells that died after coming in contact with each tracked immune cell. This tracking provides a count of immune cells that were able to invade the cell-containing hydrogel and were involved in the killing of multiple tumor cells, as well as the associated number of killed tumor cells.

In some implementations, immune serial killing analysis 340, such as reduced killing, is used as a proxy for exhaustion of immune cells.

The following experimental examples are provided to illustrate example embodiments of the present invention, and should not be considered limiting.

### EXAMPLES

### Example 1—Tumor Digestion

Tumor samples from six patients with kidney cancer were each weighed in a glass petri dish. For each sample, a 100 μm pore size cell strainer was placed on top of a 50 mL Falcon tube, and the tumor was placed in the strainer. The tumor was rinsed twice with PBS to remove any residual transport solution. A tissue plunger was inserted into one end of a Cytiva VIA Extractor processing pouch, and the tissue chunk was inserted into the pouch using forceps. Using the plunger, the tissue was pushed into the pouch. The end of the pouch was sealed using a heat sealer and 5 mL of enzyme solution (Tumor Dissociation Kit, Miltenyi Biotec) (Advanced DMEM-F12/enzyme H/enzyme R/enzyme A/Y-27632) was added to the pouch with a 5 mL luer lock syringe. The pouch was placed into the VIA extractor and the necessary dissociation program was run for 15 minutes. Once the tissue was fully digested, a 10 mL luer lock syringe was used to remove the tissue and media from the pouch. The sample was immediately strained using a 70 μm pore size strainer over a 15 mL Falcon tube. Stop solution (10 mL) was used to rinse the pouch and the mixture was strained through the same strainer. The sample was spun using a centrifuge and resuspended in medium. Red blood cells were lysed by incubating the cells in 1×RBC lysis solution for 2 minutes at room temperature. The cells were washed, spun, and resuspended in medium.

Viability of the dissociated cells was determined using a Luna cell counter.

Original sample weight and viable cell density, viability, and final volume following tissue dissociation for the six kidney tumor samples are presented below in Table 1.

TABLE 1

| | Sample weight | VCD (×10⁶ | | |
|---|---|---|---|---|
| Donor | (mg) | cells/mL) | Viability (%) | Volume (mL) |
| A | 170 | 2.14 | 91.2 | 1 |
| B | 217 | 1.25 | 82.1 | 1 |
| C | 1352 | 5.63 | 81.65 | 5 |
| D | 232 | 1.97 | 86.05 | 2 |
| E | 402 | 2.56 | 53.05 | 2 |
| F | 1413 | 2.64 | 69.2 | 3 |

### Example 2—Characterization of Tumor Culture Content

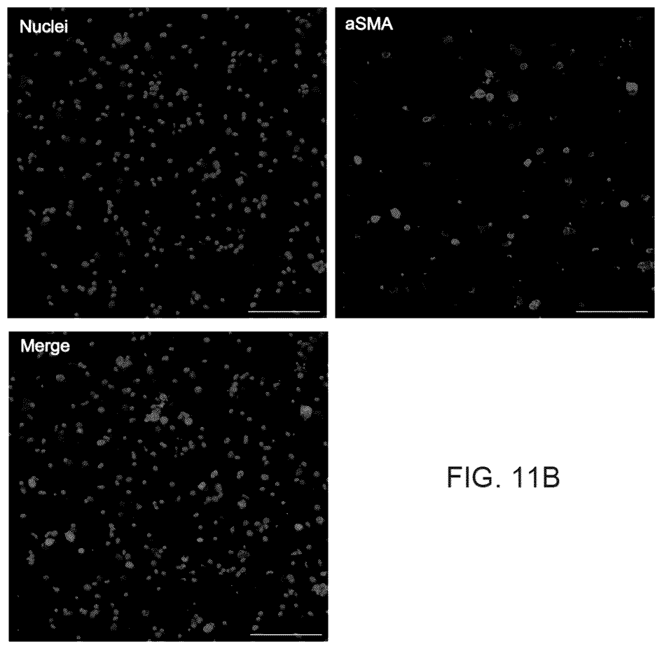

To evaluate the types and proportions of cells obtained by the disclosed methods, tumor samples from two patients were prepared as described in Example 1. Results are presented in FIGS. 11A-11E. For FIG. 11A, cells were fixed and stained with DAPI (blue), vimentin (green), and pan-cytokeratin (red). The Triple staining (i.e., blue, red, and green) in the “merge” image indicate renal cell carcinoma (RCC) cells, and double staining (i.e., blue and red) indicates epithelial cells. For FIG. 11B, cells were fixed and stained with DAPI (blue) and α-smooth muscle actin (red) to identify cancer-associated fibroblasts (blue and red). For FIG. 11C, cells were fixed and stained with DAPI (blue), CD45 (green), and CD31 (red) to identify endothelial (blue and red) and immune (blue and green) cells. FIG. 11D is a pie chart showing the distribution of various cancer and non-cancer cell types isolated from tumor tissue as determined by immunofluorescence. FIG. 11E shows flow cytometry analysis of single-stained tumor tissue-resident immune cells from one patient. Data for FIGS. 11A-11D were obtained from a single patient, and a separate patient was used to obtain data for FIG. 11E. The figures demonstrate that a diverse set of cell types could be isolated and cultured.

### Example 3—Staining and Encapsulating Dissociated Tumor Cells

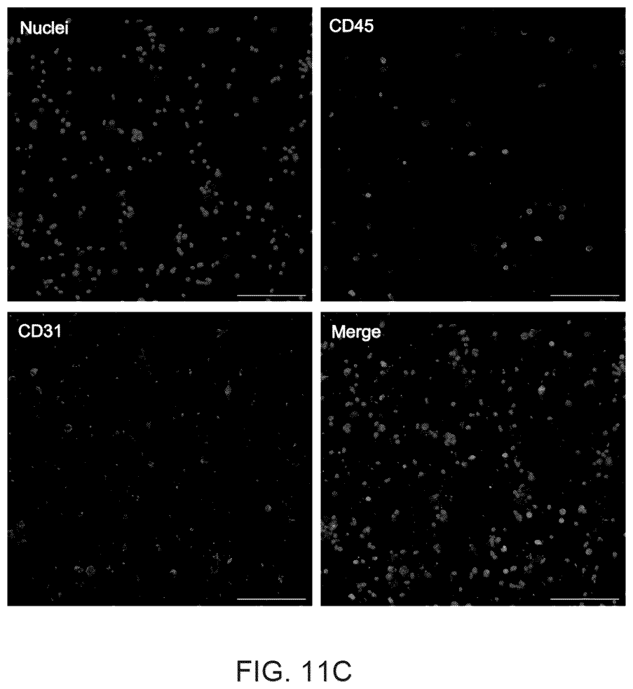

The dissociated cells prepared in Example 1 were adjusted to 1×10⁶ cells/mL and stained with an optimized concentration of a fluorescent probe that stains living cells. The cells were allowed to stain for 30 minutes at 37° C. Cells were washed with 5 volumes (i.e., 5× the initial staining volume) of culture medium, centrifuged, and resuspended at a suitable density for subsequent hydrogel encapsulation. An image of isolated, dissociated, stained tumor cells is shown in FIG. 12.

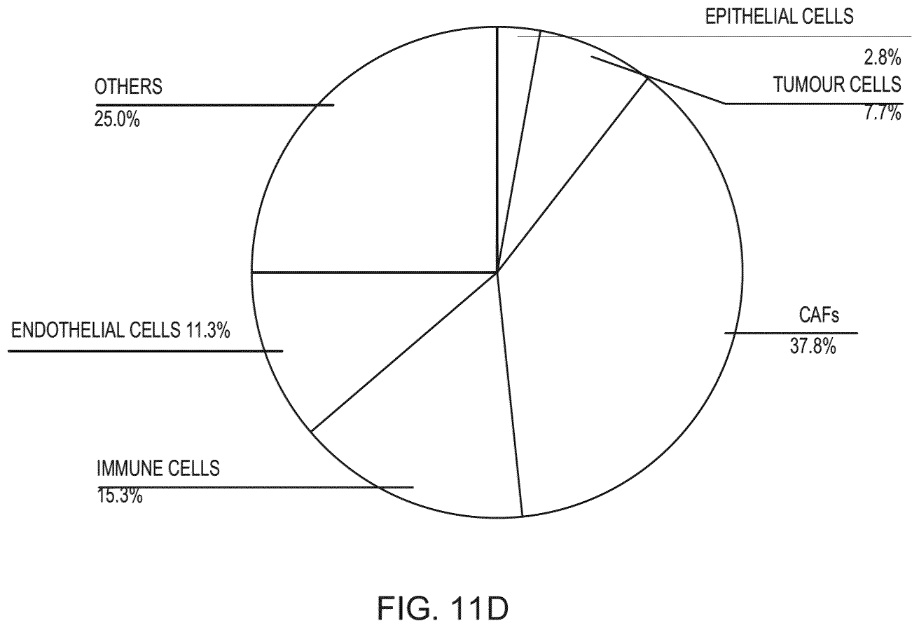

Ten thousand cells were encapsulated into a hydrogel and allowed to crosslink for less than 1 hour. FIG. 13 shows optical density (OD) (as measured by a plate reader) of crosslinking hydrogels over time for three representative experiments. Crosslinking proceeded until an OD plateau was reached.

### Example 4—Isolating Immune Cells from Whole Blood

Matched whole blood (15 mL) was diluted with an equal volume of PBS. The diluted blood was added to a LeucoSep tube by pipetting down the inner side of the tube. The LeucoSep tube was centrifuged at 950 g for 20 minutes with the brake off. The peripheral blood mononuclear cell (PBMC) layer (thin, cloudy interface between plasma and density gradient solution) was removed with a Pasteur pipette and transferred to a fresh 50 mL tube. PBS was added to the tube up to 50 mL and PBMCs were washed by spinning at 650 g for 10 minutes. The supernatant was discarded and any residual platelets were removed by adding 50 mL PBS and spinning at 290 g for 10 minutes. Residual red blood cells were lysed by incubating the cells in 1×RBC lysis solution for minutes at 37° C. The cells were washed, spun, and resuspended in medium and counted.

### Example 5—Labeling and Collecting Immune Cells

The PBMCs prepared in Example 3 were resuspended in 4° C. 1% HSA/PBS-EDTA at a density of 1×10⁷ cells/90 μL and transferred to a 15 mL tube. Ten μL of CD8 magnetic microbeads were added per 1×10⁷ cells to the same 15 mL tube and incubated for 15 minutes at 4° C. 2 mL 4° C. 1% HSA/PBS-EDTA was added to the tube. Cells were centrifuged at 300 g for 10 minutes and the supernatant was discarded. The labeled cells were resuspended in 500 μL 4° C. 1% HSA/PBS-EDTA.

For 1×10⁷ cells, one magnetic separation (“MS”) MACS column was placed onto a magnet. A tube rack with a 15 mL tube for waste was placed underneath the column. The column was prepared by rinsing with 500 μL 4° C. 1% HSA/PBS-EDTA. A new 15 mL tube was placed under the column and labeled as “Negative Fraction.” The magnetically labeled cell suspension was added to the column. Unlabeled cells that flowed through the column were collected and the column was washed with three volumes of 500 μL 4° C. 1% HSA/PBS-EDTA. The negative fraction was retained and stored on ice. A 15 mL tube labeled as “Positive Fraction” was placed in the tube rack. The MS column was removed from the magnetic separator and placed on the Positive Fraction. 1 mL 4° C. 1% HSA/PBS-EDTA was added to the column and the bound cells were immediately flushed through the column with the column plunger. Both the Negative Fraction and Positive Fraction were counted, washed, and resuspended in PBS.

### Example 6—Staining Subsets of Immune Cells

The Positive Fraction (CD8⁺ PBMCs) prepared in Example 4 was adjusted to 2×10⁶ cells/mL and stained with an optimized concentration of an amine-reactive fluorescent probe that emits at 450 nm. The Negative Fraction (CD8⁻ PBMCs) was adjusted to 2×10⁶ cells/mL and stained with an optimized concentration of a thiol-reactive fluorescent probe that emits at 650 nm. Cultures were allowed to stain for 30 minutes at 37° C. Cells are washed with 5 volumes of culture medium (i.e., 5× the volume in which they were initially stained), centrifuged, resuspended in culture medium, and combined with one another. The culture was then counted and the density was adjusted for subsequent processing.

### Example 7—Activating Immune Cells

A culture vessel was coated with a 1 μg/mL αCD3 solution in PBS. The vessel was incubated at 37° C. for 2 hours. The solution was removed and PBMCs from Example 6 and both the Positive and Negative Fractions were added at a final density of 1×10⁶ cells/mL in TexMACS™ containing 5% HSA and 120 U/mL IL-2. The cells were harvested less than 24 hours later by transferring the supernatant to a 50 mL tube. Residual cells were washed from the culture vessel using PBS. Any adhered cells were removed with an appropriate volume of TrypLE and incubated for 10 minutes at 37° C. Culture medium (5 volumes) was added and combined with the culture in a 50 mL tube. Cells were centrifuged and resuspended in an appropriate volume of culture medium.

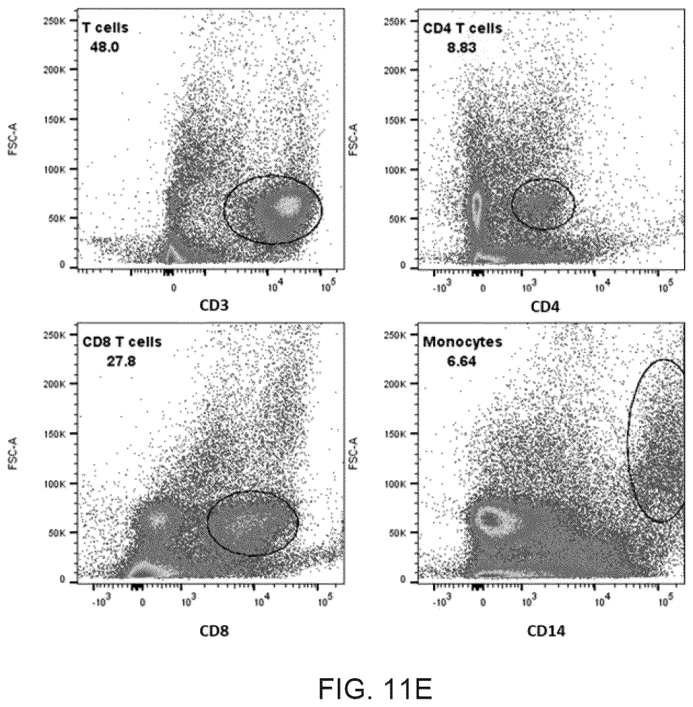

T cell activation was confirmed by measuring fluorescence intensity of the CD95 channel. Results are presented in FIG. 14 as the effect of CD95 expression in un-activated (solid histogram) and CD3-activated PBMCs (dashed histogram) on CD4⁺ (14A) or CD8⁺ (14B) T cells. Each histogram is displayed relative to mode to account for differences in cell number between samples; the X axis is CD95 fluorescence and the Y axis is Relative Signal (%).

### Example 8—Adding Immune Cells to Hydrogels

The concentration of PBMCs from Example 7 was adjusted to 5×10⁵ cells/mL. The PBMC culture (100 μL) was added to 100 μL of TexMACS™ medium containing 5% HSA and 120 U/mL IL-2 on top of the tumor cell-containing hydrogel prepared in Example 3. The PMBCs were added within 24 hours of the tumor cells being incorporated into the hydrogel. The final tumor-immune culture contained about 50,000 PBMCs and about 10,000 tumor cells.

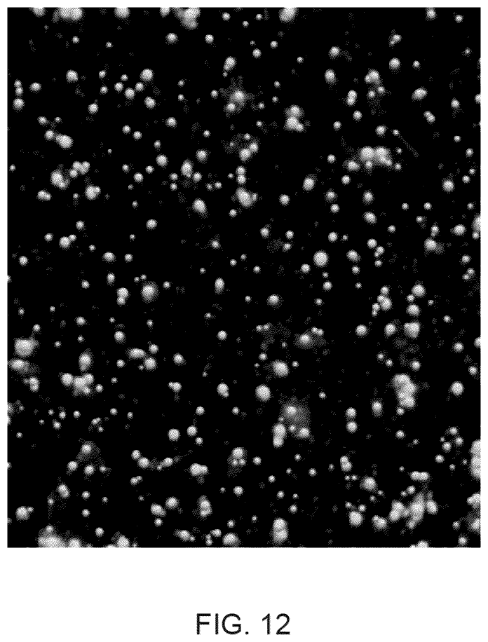

A comparison of cultures is shown in the maximum intensity projections of FIGS. 15A-15C in which 10,000 tumor cells were encapsulated in hydrogels and cultured on their own (FIG. 15A), with 50,000 PBMCs (FIGS. 15B & 15D), or with 100,000 PBMCs (FIG. 15C). PBMCs were added to the culture supernatant and formed a boundary/interface between the hydrogel and culture medium as visualized in the 3D perspective of FIG. 15D. Tumor cells, CD8⁺, and CD8⁻ cells are indicated by yellow, blue, and red, respectively.

### Example 9—Adding Dead Cell Dyes

A dead cell dye cocktail was prepared with three constituents. Constituent A was impermeable to intact membranes and could detect cells undergoing early apoptosis, but was also able to detect cells undergoing later stages of apoptosis or necrosis. Constituent B was membrane-permeable and could detect cells in the early and late apoptotic states. Constituent C was membrane-impermeable and could detect cells in the late apoptotic or necrotic state. An optimized volume of each constituent was added to culture medium in a 1.5 mL Eppendorf tube to create a 200×concentrate with respect to each constituent. 1 μL of the cocktail was added to the tumor-immune culture immediately after addition of the PBMCs as described in Example 8. Cultures were allowed to stain for 30 minutes at 37° C.

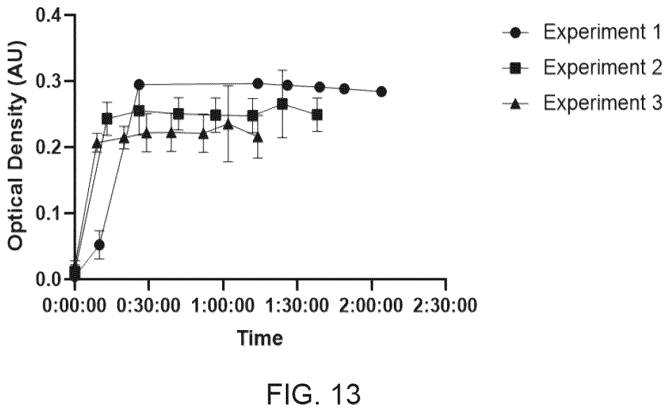

An example culture is shown in the maximum intensity projections of FIGS. 16A-16D in which 10,000 tumor cells were encapsulated in hydrogels and stained with Constituent A (FIG. 16A), Constituent B (FIG. 16B), Constituent C (FIG. 16C), or all three constituents together (FIG. 16D). All constituent dyes exhibit green fluorescence.

### Example 10—Baseline Imaging

Tumor-immune cell cultures were placed in the imaging platform of a Leica Stellaris confocal microscope. Laser intensity and gain were adjusted for optimal brightness and least amount of spillover between channels. Each fluorescence channel was scanned (sequentially or simultaneously) from the bottom to the top of the culture.

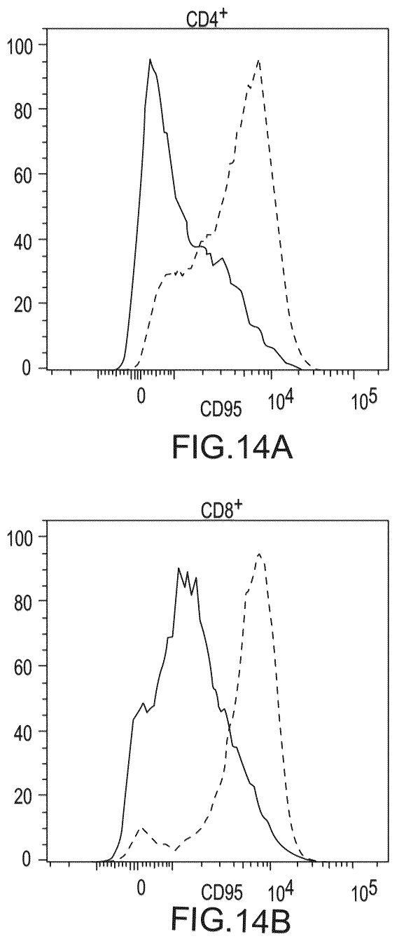

An example culture, produced from 10,000 tumor cells encapsulated in a hydrogel and cultured with 50,000 PBMCs, is shown in the maximum intensity projection of FIG. 17A. Tumor cells, CD8⁺, and CD8⁻ cells are visualized by yellow, blue, and red respectively. Green areas indicate cell death. Corresponding cell counts are shown in FIG. 17B.

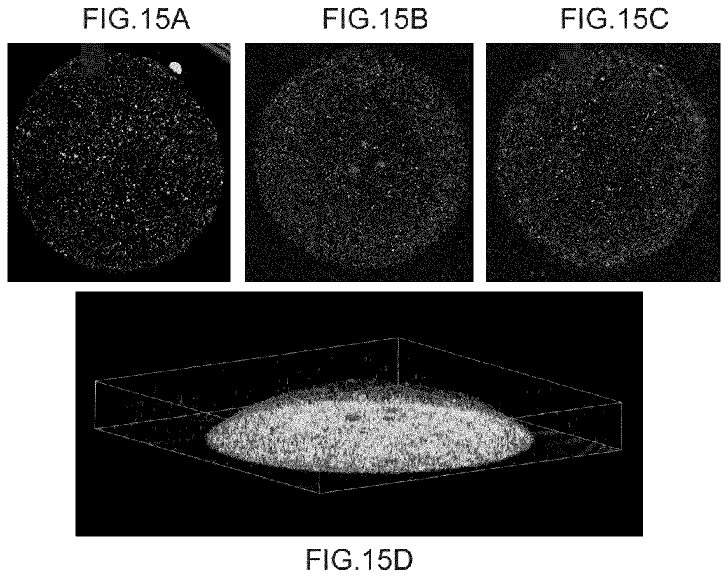

Another example culture is shown in FIG. 18, which is a 3D volume rendering view of an immune-tumor co-culture. Tumor cells, CD8+, CD8− are visualized by yellow, blue, and red respectively. Green areas indicate cell death.

### Example 11—Adding a Test Agent

The cell culturing and monitoring methods disclosed herein permit investigation of the effects of various test agents on a tumor-immune culture.

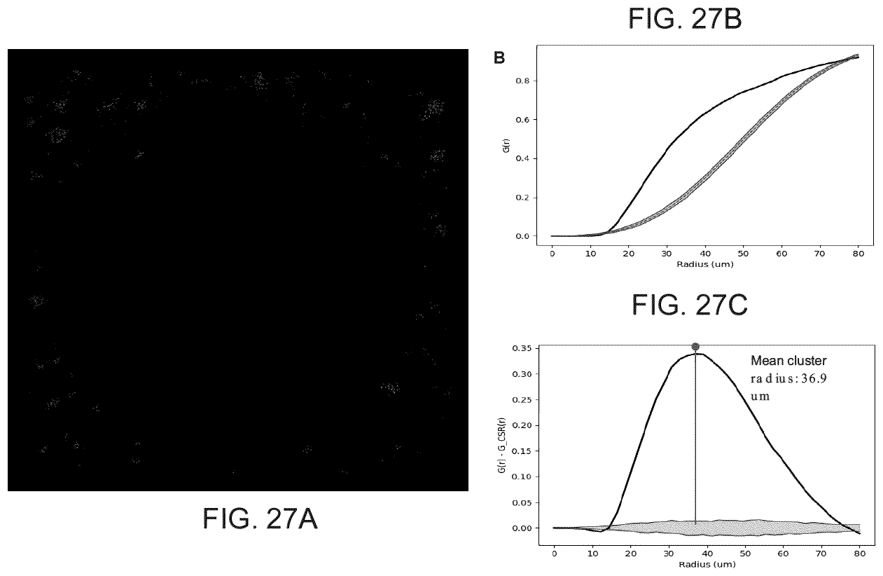

A) An aliquot of pembrolizumab (25 mg/mL) was diluted in PBS to achieve a working stock of 1 mg/mL. 2 μL of this stock is added to the tumor-immune culture to achieve a final dose of 10 μg/mL. In parallel, 2 μL of a 1 mg/mL stock of human anti-IgG4 is added to a negative control tumor-immune culture. A representative experiment is further described in Example 17 and FIG. 31-36.

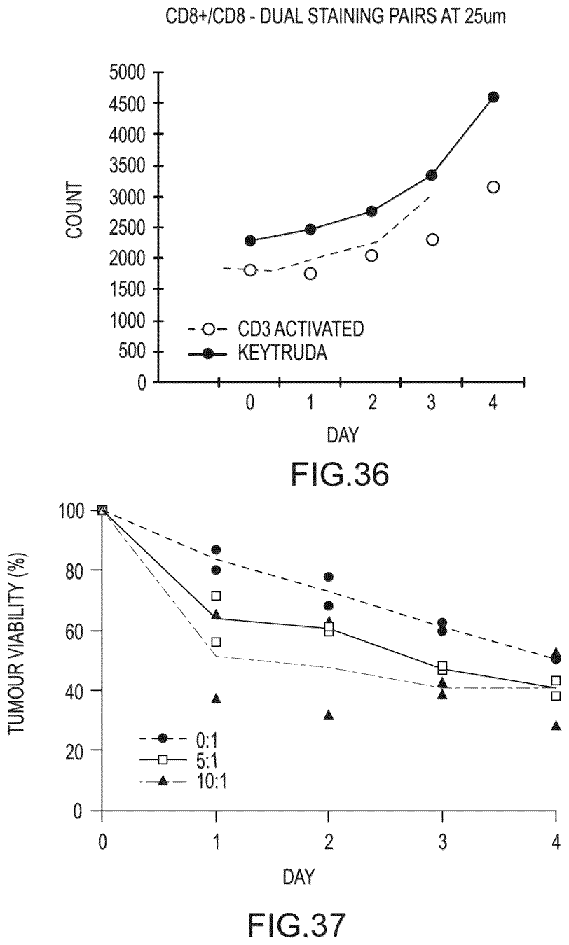

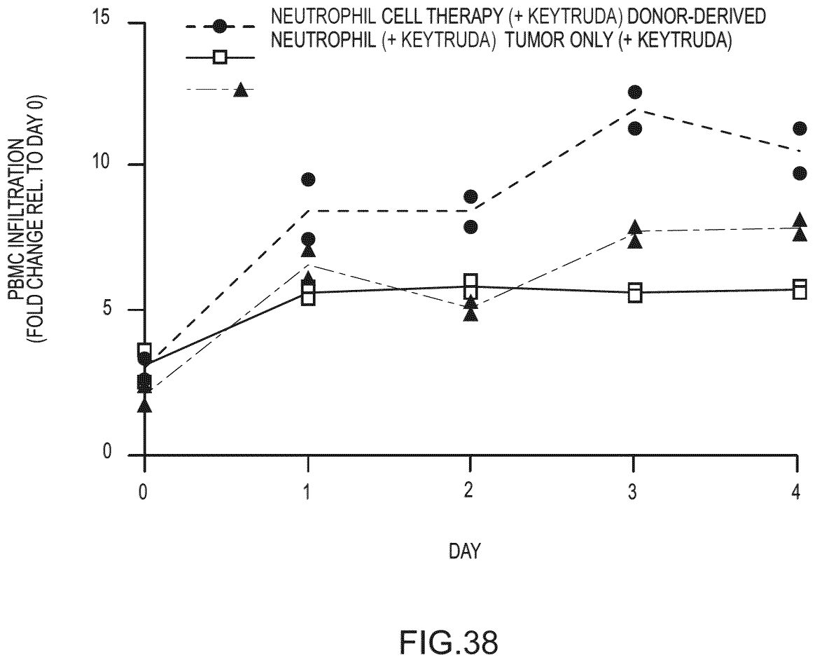

B) Fifty thousand stained cells of an allogeneic neutrophil cell therapy were added to the tumor-immune culture, either by combining the neutrophils with the tumor cells in a hydrogel, or by adding the neutrophils with the immune cells onto the tumor-containing hydrogel. A representative experiment is further described in Example 18 and FIG. 37-38.

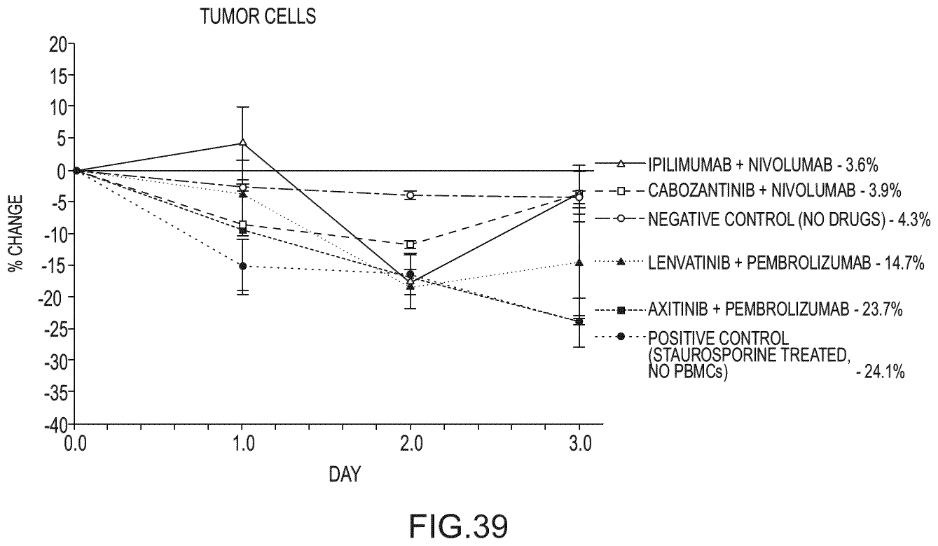

C) Eighteen immune-tumor cultures were created from the same patient sample to test 6 conditions in triplicate. The conditions were as follows: negative control (no treatment), positive control (staurosporine (10 μM)), axitinib (10 μM)+pembrolizumab (10 μg/mL) treatment, lenvatinib (10 μM)+pembrolizumab (10 μg/mL) treatment, cabozantinib (10 μM)+nivolumab (10 μg/mL) treatment, and ipilimumab (10 μg/mL)+nivolumab (10 μg/mL) treatment. A representative experiment is further described in Example 19 and FIG. 39.

### Example 12—Imaging Over Time

Cultures were placed in the imaging platform of a Leica Stellaris confocal microscope. Laser intensity and gain were adjusted for optimal brightness and least amount of spillover between channels. Each fluorescence channel was scanned from the bottom to the top of the culture. A whole culture was imaged every 60 minutes for 16 hours. Another culture was imaged every 24 hours for 4 days.

Example images are shown in FIGS. 11 & 12. In FIG. 11, the images are from a 56 μm section of a live cell time-lapse recorded at 1-hour intervals over 16 hours. The tumor cell indicated by an arrow can be seen interacting with CD8⁺ (blue) and CD8⁻ (red) cells at T2, T4, and T8 before staining positive with the dead cell dye cocktail (green) at T13.

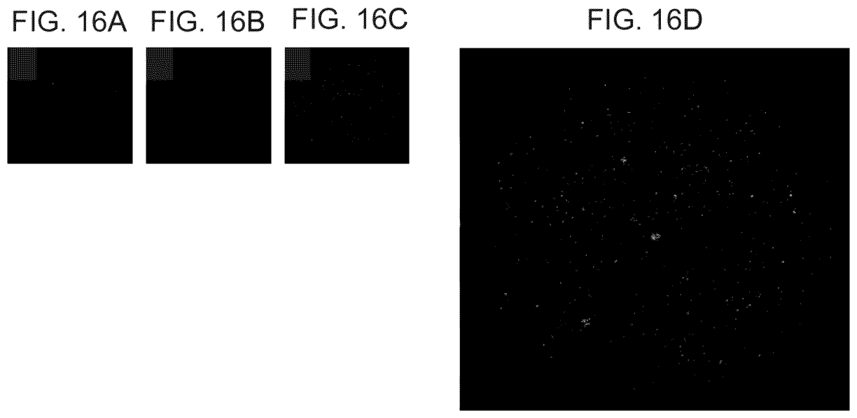

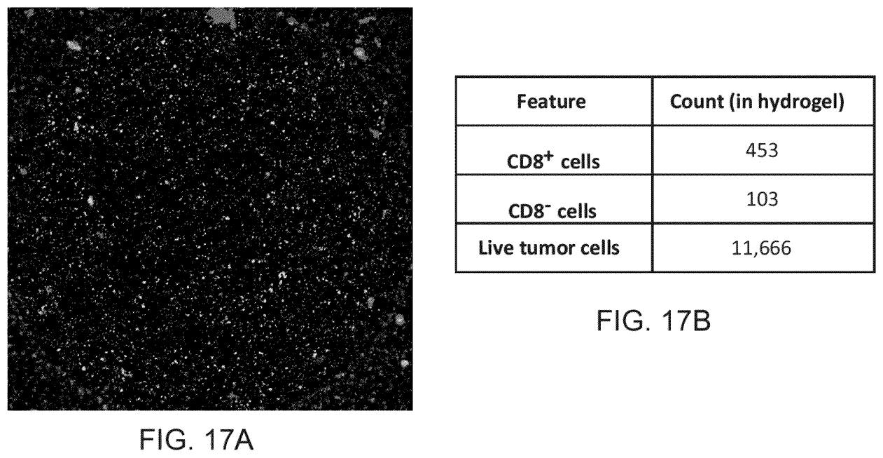

In FIG. 20, the images, which were produced after processing a tumor from a different patient than the patient of FIG. 19, are from maximum intensity projections of a culture produced from 10,000 tumor cells encapsulated in a hydrogel, cultured with 50,000 PBMCs, and monitored over 4 days. Tumor cells, CD8⁺, and CD8⁻ cells are shown in yellow, blue, and red respectively. Dead cells were stained green upon death or initiation of cell death pathways.

### Example 13—Measuring Levels of Soluble Factors

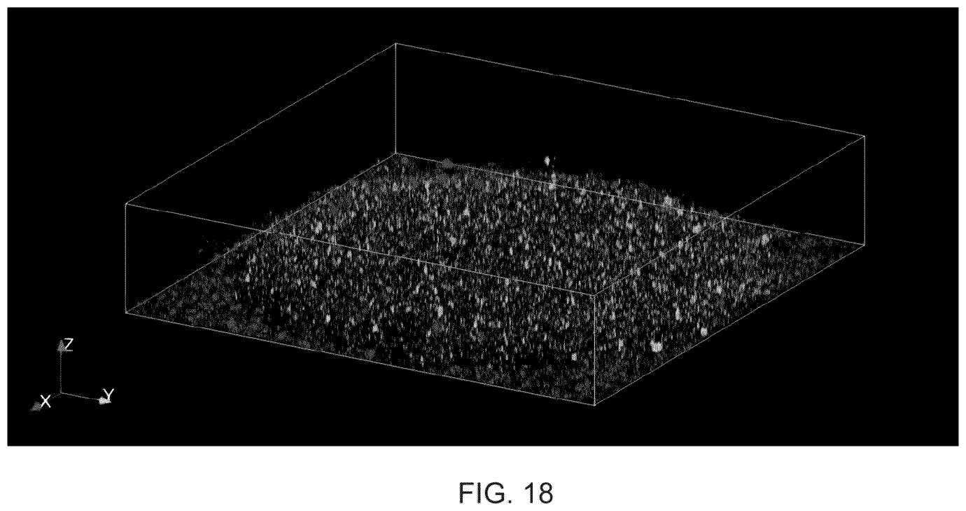

PBMCs from a renal cell carcinoma patient's blood sample were processed and cultured according to the disclosed methods and in the presence of a receptor tyrosine kinase inhibitor—axitinib (1 or 10 μM), lenvatinib (1 or 10 μM), cabozantinib (“cabo.”) (1 or 10 μM)—or DMSO as a control. For each of culture day 1, 2, and 3 (at which point the culture was terminated) 200 μL of the culture supernatant was removed from the sample vessels/wells of interest. Samples were spun at 400 g for 105 minutes to remove any intact cells. The supernatant was collected, transferred to a fresh tube and spun at 12,000 g for 10 minutes to remove any cellular debris or precipitate. The supernatant was collected, aliquoted into 50 μL aliquots, and frozen for subsequent analysis. Samples were diluted 1:5 in 1× assay diluent. Samples were processed and analyzed by sandwich ELISA for TNFα according to the manufacturer's guidelines (BioTechne). Results are shown in FIG. 21. Dotted lines show the maximal production of cytokines relative to the control sample on each specific day. FIG. 21 demonstrates successful multi-timepoint testing of soluble factors.

### Example 14—Terminating Cell Cultures

In various experiments, each of the following post-culture termination methods was used to further analyze the cell culture response.

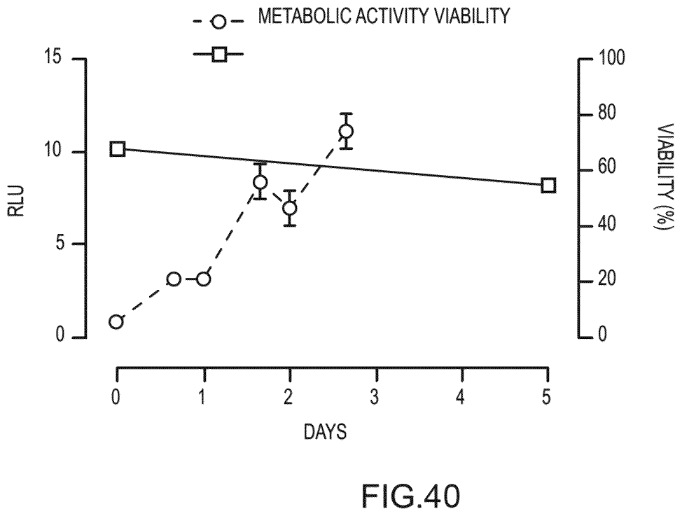

Metabolic activity measurement: Following encapsulation, 150 μL of pre-warmed medium containing 1×MT cell viability substrate and 1×NanoLuc Enzyme were slowly added on the border of the well to avoid detachment or broken gel droplets. An equal volume of medium containing 1×RTG reagents was also added into 3 blank wells as a control. The plate was covered with foil and placed on a rocker for 5 minutes at room temperature. The plate was incubated for 2 hours at 37° C. The temperature of a plate-reader was set to 37° C., the plate was inserted into the reader, and the luminescence was measured. Each plate was scanned every morning and afternoon, i.e. at 16 hours, 24, 40, 48, 64, and 72 hours after addition of RTG reagent. A representative experiment is further described in Example 19 and FIG. 40.

Cell viability fluorescence endpoint: The media was removed from the wells of interest and a Calcein AM/Ethidium homodimer III solution was added to the wells of interest, in accordance with the manufacturer's instructions. The plate was incubated for 37° C. for 30 minutes and imaged by confocal microscopy. A representative experiment is further described in Example 19 and FIG. 40. Other experiments (not shown) have demonstrated the viability of the tumor-immune cell culture prepared by the disclosed methods to be up to 14 days.

RNA measurement: All surfaces of the workstation were wiped with RNase-ZAP. Using a sterile spatula, three hydrogels were removed from their lodging and placed in a single gentleMACS M tube. 600 μL TRIzol was added to the hydrogels directly and an optimized program on the gentleMACS dissociator was run twice. The tube was centrifuged at 500 g for 20 seconds after each dissociation cycle. After the dissociation procedure, full homogenization of the hydrogels was ensured. The dissociation process was repeated when the hydrogels were not fully homogenized. The digest was transferred to a new RNAse-free tube. Chloroform was added to the digest at a ratio of 1:5 (chloroform: digest volume) and mixed by inverting the tube several times. The tube was incubated at room temperature for 5 minutes and centrifuged at 21000 g for 15 minutes at 4° C. The aqueous phase was transferred to a QIAshredder spin column placed in a 2 ml collection tube. The columns were centrifuged for 2 minutes at 21,000 g. The flow through was retained and an equal volume (not exceeding a combined volume of 700 μL) of 70% ethanol (molecular grade, sterile, RNAse-free) was added.

The sample was transferred to an RNeasy MinElute column and placed in a 2 mL collection tube. The tube was centrifuged for 15 seconds at 21,000 g and the flow-through was discarded. Three hundred fifty μL Buffer RW1 was added to the column, the column was centrifuged for 15 seconds at 21,000 g and the flow-through was discarded. Ten UL DNase I was added to 70 μL Buffer RDD and mixed. This solution was added to the column membrane and incubated at room temperature for 15 minutes. Three hundred fifty μL Buffer RW1 was added to the column, centrifuged for 15 seconds at 21,000 g and the flow-through was discarded. The column was transferred to a new collection tube, 500 μL of 70% ethanol was added, and the column was centrifuged for 15 seconds at 21,000 g with the flow-through. The column was transferred to a new collection tube and centrifuged at 21,000 g for 2 minutes. The column was transferred to a 1.5 mL Eppendorf tube, 14 μL of RNase-free water was added to the column membrane, and the column was centrifuged at 21,000 g for 1 minute.

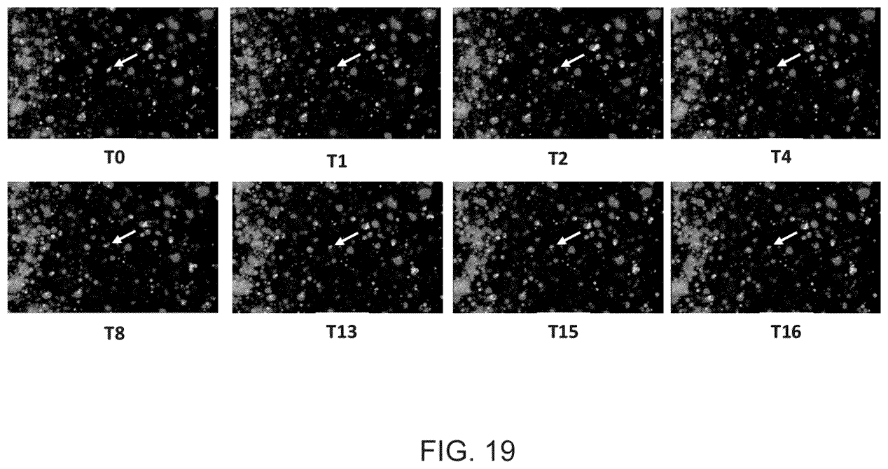

An RNAseq analysis of transcripts from 3D cell cultures (Day=3) formed from a collection of triple-negative breast cancer patient tumor samples compared against tumor cell pellets is shown in FIG. 22. FIG. 22A is a principal component analysis (PCA) of mRNA transcripts from six patients after three days of culture in hydrogel formulation compared to cell pellets from the same patient; FIG. 22B is a volcano plot of differentially expressed genes in pellet vs hydrogels; and FIG. 22C shows gene ontology analysis of hydrogel cultures vs pellets.

### Example 15—Computer Vision Metrics

A tumor and matched blood sample from a patient with renal cell carcinoma were prepared and cultured according to the foregoing examples. The culture was evaluated for numerous metrics according to the methods disclosed herein and without disrupting the integrity of the 3D cell culture.

Immune cell migration was investigated as described for cell migration analysis 332 in method 300. A one-hour time-lapse with a four-minute interval between each frame was performed and the results are shown in FIGS. 23A-23B. CD8⁺ cells (FIG. 23A) and tumor cells (FIG. 23B) were tracked by solving the linear assignment problem and the average migration speed was calculated.

Immune cell infiltration of the tumor-derived hydrogel culture was investigated as described for immune cell infiltration analysis 336 in method 300. Results are shown in FIG. 24A, in which tumor cells (in yellow) are used to detect the interface (in white) between the hydrogel and culture medium. FIG. 24B shows the total detected CD8⁻ immune cells (in red) and FIG. 24C shows the infiltrated CD8⁻ immune cells. Associated counts are shown in FIG. 24D.

Death of immune cells and tumor cells from the tumor-derived hydrogel culture were investigated as described for immune cell and tumor cell death analysis 342 in method 300. Results for immune cells are shown in FIG. 25. FIG. 25A shows a maximum intensity projection of CD8⁻ immune cells (in red) and dead cell dye (in green). FIG. 25B is a chart showing cell counts of CD8⁻ cells, all dead cells, and dead CD8⁻ cells, obtained by dye co-localization. Results for tumor cells are shown in FIG. 26. FIG. 26A shows a maximum intensity projection of tumor cells (in yellow) and dead cell dyes (in green). FIG. 26B is a chart showing cell counts of tumor cells, all dead cells, and dead tumor cells, obtained by dye co-localization.

Immune cell clustering from the tumor-derived hydrogel culture was investigated as described for cell clustering analysis 338 in method 300. Results are shown in FIG. 27. Spatial cell clustering analysis was performed on CD8⁻ cells, using Ripley's G function. FIG. 27A shows clusters of cells detected using the DBSCAN algorithm, obtained for a characteristic clustering radius of ˜37 μm and labeled with the same colors. Cells colored in dark blue are sparsely distributed at this clustering radius and do not belong to any cluster. The characteristic clustering radius was obtained from Ripley's G function.

FIG. 27B shows the observed Ripley's G function (black solid line), while the blue line corresponds the expected G function for a Poisson process representing complete spatial randomness (CSR) and the blue shaded area is the Monte Carlo envelope that corresponds to the 5th to 95th percentiles of CSR. Ripley's G functions are plotted against the characteristic clustering radius.

In FIG. 27C, the observed G function (black solid line) and expected G function for CSR (which is a constant line at zero, and is shown with an envelope representing the confidence interval—i.e., the range of G function values expected to be observed under the null hypothesis) are plotted on the same graph. If the observed G function is above the expected G function, it indicates clustering, and if it is below, it indicates dispersion at a particular clustering radius.

The clustering radius is identified by searching positive peaks in FIG. 27C, where the observed function deviates significantly from zero (G function of CSR). The Monte Carlo envelope is used as a statistical test to determine if the observed patterns deviate in a statistically significant way from complete spatial randomness. The red vertical line shows the clustering radius of ˜37 μm, which was used to determine cell clusters in FIG. 27A.

Another example of investigating immune cell clustering from the tumor-derived hydrogel culture, as described for cell clustering analysis 338 in method 300, is provided in FIG. 28. Clustering was measured between CD8⁺ immune cells and CD8⁻ immune cells using bivariate pair-correlation analysis. Whereas FIGS. 28A-B show bivariate spatial association of two classes of cells (i.e., CD8⁺ and CD8⁻), FIGS. 27A-C described above show univariate clustering of a single class of cells (i.e., CD8⁻). Also, FIGS. 28A-B use bivariate pair-correlation function and FIGS. 27A-C use Ripley's G function. FIG. 28A is a maximum intensity projection of the CD8⁻ immune cells (in red) and CD8⁺ immune cells (in blue). In the bivariate pair-correlation analysis of FIG. 28B, the solid (black) line corresponds to the pair-correlation function, while the dashed (blue) line corresponds to a Poisson process representing complete spatial randomness (CSR) and the shaded surrounding area is the Monte Carlo envelope that corresponds to the 5th to 95th percentiles of CSR. Pair-correlation analysis shows a statistically significant spatial correlation between CD8⁻ immune cells and CD8⁺ immune cells at radii distances between 8 and 33 μm.

Immune-tumor cell engagement from the tumor-derived hydrogel culture was investigated as described for immune-immune, tumor-immune cell contact analysis 344 in method 300. Results are shown in FIG. 29, in which tumor cells are visualized in yellow, CD8⁻ immune cells in red, and CD8⁺ immune cells in blue. The red circle highlights an area of immune-tumor cell engagement.

### Example 16—Computer Vision Metric Example: Tumor Cell Count

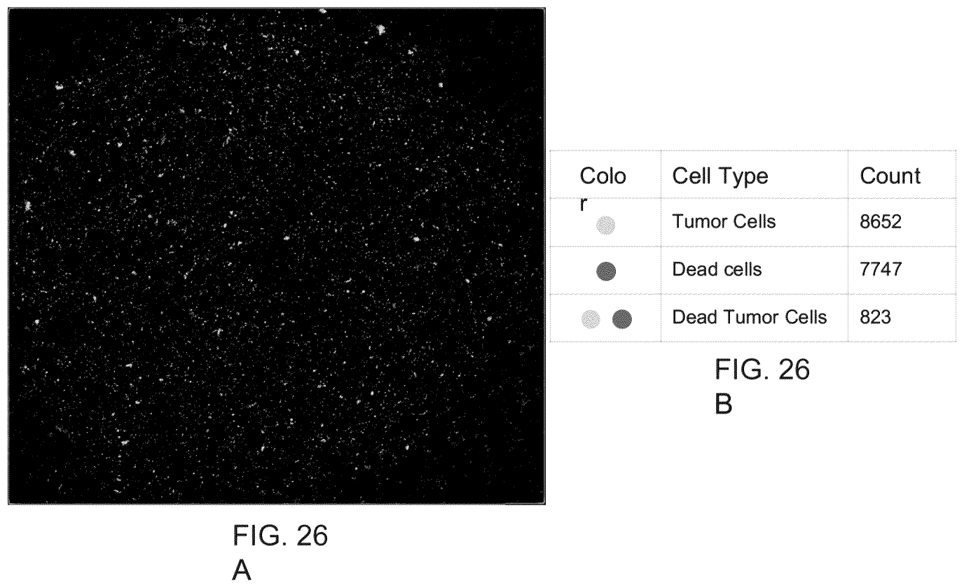

Immune-tumor co-cultures were formed according to the foregoing Examples using a tumor sample obtained from a renal cell carcinoma patient. Cultures were treated with axitinib (10 μM)+pembrolizumab (10 μg/mL) or were not treated (negative control). Tumor cell numbers were counted as described above. Results are shown in FIG. 30 as percent change of tumor cell count over time. Treated cultures showed a statistically significant difference in cell count when compared to the negative control cultures within 24 hours. Specifically, a reduction of 17% in tumor cell count was observed in the treated sample, while the negative control showed a reduction of only 3.9%. Two replicates were used for each condition. The error bars represent the standard deviation of the two replicates.

### Example 17—Comparing Keytruda Treatment to CD3 Activation

To demonstrate the effectiveness and reliability of the disclosed cell culturing and analysis methods, a known chemotherapeutic, Keytruda (pembrolizumab), was tested. A tumor sample and matched blood sample from a patient with renal cell carcinoma were prepared according to the foregoing Examples and methods. Briefly, PBMCs were isolated from blood and CD8⁺ and CD8⁻ cells were separated, stained, and recombined as previously described. PBMCs were either cultured with 1 μg/mL anti-CD3 or 10 μg/mL Keytruda for 18 hours. Tumors were dissociated and stained and 10,000 cells were encapsulated in hydrogel as previously described. At TO, 100,000 PBMCs from each condition were added to tumor cultures. Live cell imaging, using confocal microscopy, was conducted each day for 5 days and images were analyzed using the disclosed computer vision pipeline. Results are presented in FIGS. 31-36. The presented data are from single replicates obtained from one experiment.

FIG. 31 shows absolute counts of infiltrated CD8⁺ PBMCs in 3D tumor-immune cultures. CD8⁺ PBMCs were tracked over 5 days by live cell imaging of the entire 3D tumor-immune culture. The images from each timepoint were processed and analyzed to obtain counts of infiltrated cells. The results demonstrate increased infiltration of CD8⁺ cells into the tumor-containing hydrogel in the Keytruda-treated sample compared to the CD3-activated sample, which is evident as early as Day 1.

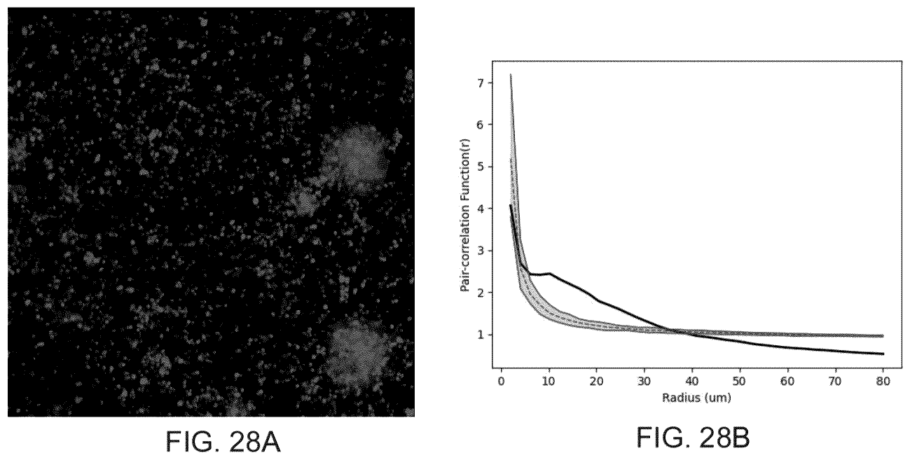

FIG. 32 shows the viability of tumor cells in the 3D tumor-immune culture. The viability of tumor cells cultured with PBMCs activated with CD3 or treated with Keytruda was tracked over 5 days by live cell imaging of the entire 3D tumor-immune culture. The images from each timepoint were processed and analyzed to obtain counts of viable cells. The results demonstrate increased tumor cell death in the Keytruda-treated sample compared to the CD3-activated sample, which is evident by Day 2.

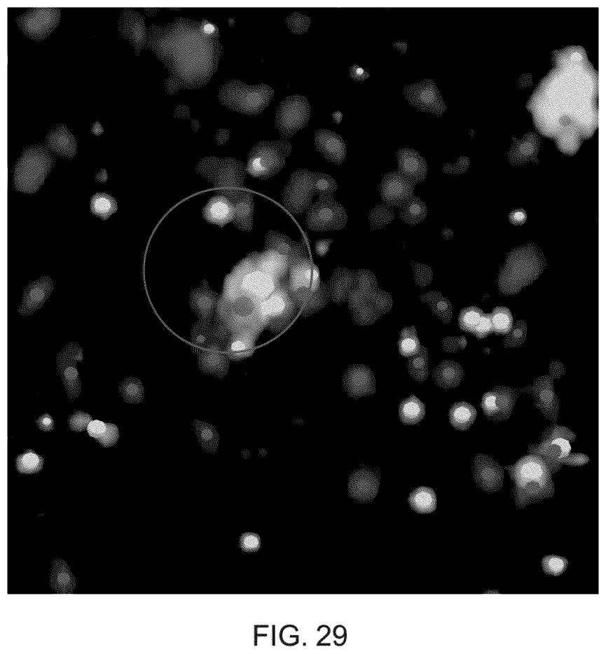

FIG. 33 shows the speed of CD8⁺ PBMCs infiltrated into the 3D tumor-containing hydrogel culture. The speed of CD3-activated or Keytruda-treated CD8⁺ PBMCs in the tumor-containing hydrogel was tracked over 5 days by live cell imaging. A region of interest was acquired each day by timelapse imaging (30 frames at 2-minute intervals). The images from each interval were processed, analyzed, and each cell's journey tracked to obtain the measure of speed over time. The results demonstrate a higher CD8⁺ cell migration peak speed in the Keytruda-treated sample compared to the CD3-activated sample.

FIG. 34 shows 3D tumor-immune culture dome size. The dome size of microtumors cultured with CD3-activated or Keytruda-treated PBMCs was tracked over 5 days by live cell imaging of the entire 3D tumor-immune culture. The images from each timepoint were processed and analyzed to obtain the volume of the tumor-containing hydrogel dome. The results demonstrate that dome volume shrinks more for the Keytruda-treated sample compared to the CD3-activated sample, which is evident by Day 1. The rate of dome volume shrinking is also higher at Day 1.

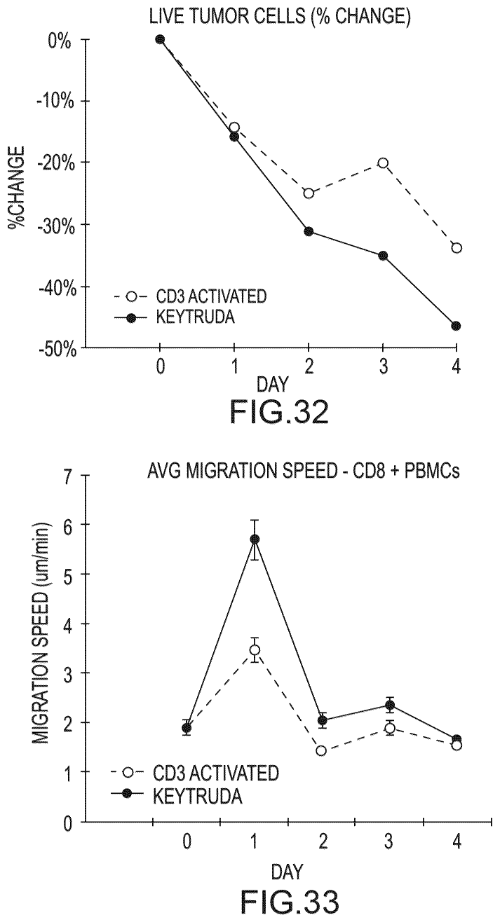

FIG. 35 shows clustering of hydrogel-infiltrated CD8⁺ PBMCs. The clustering of CD3-activated or Keytruda-treated CD8⁺ PBMCs was measured over 5 days by live cell imaging. The entire 3D tumor-immune culture was imaged at each timepoint. The images from each timepoint were processed and analyzed for the number of clusters present. The results demonstrate that CD8⁺ cells associated with each other sooner when activated with CD3, but Keytruda treatment resulted in more clustering over time.

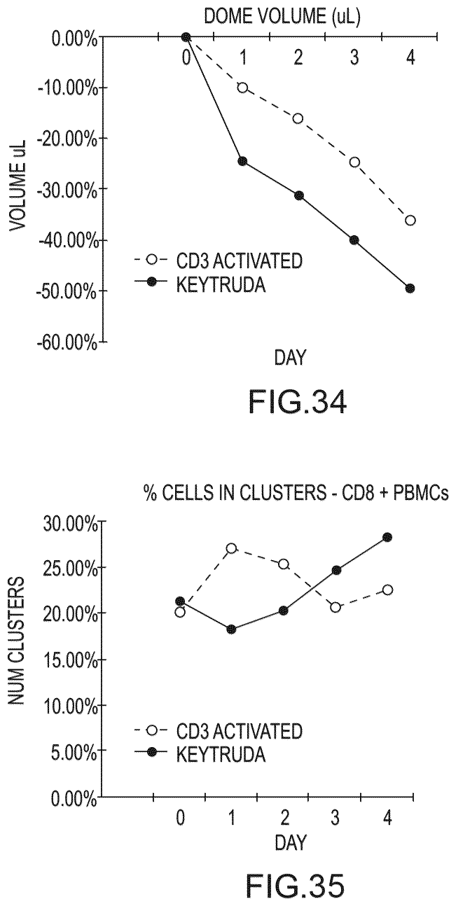

FIG. 36 shows clustering of hydrogel-infiltrated CD8⁻ and CD8⁺ PBMCs. The clustering of CD3-activated or Keytruda-treated CD8⁻ and CD8⁺ PBMCs was measured over 5 days by live cell imaging. The entire 3D tumor-immune culture was imaged at each timepoint. The images from each timepoint were processed and analyzed for the number of clusters present. The results demonstrate that CD8⁺ and CD8⁻ cells clustered together more with Keytruda treatment than with CD3 activation throughout the course of the experiment.

### Example 18—Testing Novel Cell Therapy and Combination Therapies

In this example, tumor cells were dissociated and stained as previously described. PBMCs were isolated from blood and stained with a single cell-tracking dye as previously described. In parallel, a neutrophil cell therapy was stained with a different cell tracking dye. Both the stained tumor cells and neutrophil cell therapy were counted and appropriate numbers of cells from each fraction were combined to create new samples comprising a set number of neutrophils and tumor cells at a specific ratio to one another (0:1, 5:1 or 10:1). In some experiments (see FIG. 38, non-therapeutic neutrophils derived from the patient being tested were used as a control for the cell therapy. The cell therapy-tumor co-cultures were encapsulated in hydrogels, then PBMCs were added to the cultures as previously described. To ascertain the impact of the neutrophil cell therapy on PBMC infiltration and tumor killing, the cultures were subjected to live cell imaging over the course of 5 days as previously described. Results are presented in FIGS. 37 & 38. The presented data are from duplicate samples.

FIG. 37 shows the impact of neutrophil cell therapy on tumor viability. A higher ratio of cell therapy (i.e., 10:1 compared to 5:1 or 0:1) results in higher tumor killing.

FIG. 38 shows the impact of neutrophil cell therapy on tumor viability. Tumor cells were co-encapsulated with cell therapy neutrophils or donor-derived, unmodified neutrophils. All groups received Keytruda (pembrolizumab) (10 μg/mL). The neutrophil-tumor-immune co-cultures were monitored by live cell imaging over 5 days and PBMC infiltration was determined at each timepoint. The results demonstrate that neutrophil cell therapy results in better immune cell infiltration than unmodified autologous neutrophils, which was comparable to control (tumor only, Keytruda treated).

### Example 19—Testing Multiple Therapies to Compare Efficacy

In this example, a kidney tumor sample was dissociated and extracted cells were stained as previously described (all tumor-dissociated cells were stained color 1). PBMCs were isolated from blood, stained with two cell-tracking dyes (color 2 for the CD8⁺ cell fraction, color 3 for the CD8⁻ cell fraction), and activated as previously described. 3D co-cultures were formed as previously described (1:5 tumor:PBMCs, 5K:25K cells). A dead cell dye cocktail (3 dyes, all color 4) was added as previously described. To quantify the differential effects of treatment combinations on PBMC infiltration and tumor killing, the cultures were subjected to live cell imaging over the course of 3 days as previously described. A positive control (Staurosporine, 5 μM) and a negative control (no treatment) were also evaluated. First-line combination therapies comprising immunotherapies (ipilimumab, nivolumab, pembrolizumab) and receptor tyrosine kinase inhibitor therapies (cabozantinib, lenvatinib, axitinib) for metastatic renal cell carcinoma were tested. For each condition, n=3 tumor-immune cultures were tested.

Results are presented in FIG. 39, which shows changes in tumor cell numbers as quantified through the disclosed computer vision pipeline over 3 days of tumor-immune co-culture, normalized to Day 0. The 3D cultures were treated with ipilimumab (10 μg/mL), nivolumab (10 μg/mL), pembrolizumab (10 μg/mL), cabozantinib (10 μM), lenvatinib (10 μM), and axitinib (10 μM) in the stated combinations and compared to negative (no treatment) and positive (staurosporine, 5 μM) controls. Percent change in the number of tumor cells at Day 3 is presented in the figure.

Staurosporine, the effective but highly toxic compound, decreased tumor cell count the most, as expected of a positive control, and thereby validated the accuracy and reliability of the disclosed methods. Axitinib+pembrolizumab performed almost as well and is an FDA-approved treatment combination.

### Example 20—Testing Metabolic Activity and Cell Viability

Metabolic activity and cell viability of untreated tumor cells in a hydrogel were evaluated. Metabolic activity was tested using an NAD/NADH-Glo Promega assay according to the manufacturer's instructions. Results are presented in FIG. 40 in relative light units (RLU; left axis; open circles). The results show that metabolic activity in the tested culture increased over time for three test days.

Cell viability was measured by a Live/Dead Invitrogen endpoint assay according to the manufacturer's instructions on Day 0 (pre-encapsulation) and on Day 5 (right axis; open squares). Note that the culture on Day 0 is different from the culture on Day 5 for the viability assay because it is not a live-cell assay. The present assay validated the ability of the disclosed culturing methods to produce a viable culture for at least five days. Specifically, cell viability in the disclosed system does not decrease significantly from baseline for at least five days.

Although various representative embodiments and implementations have been described above with a certain degree of particularity, those skilled in the art could make numerous alterations to the disclosed embodiments without departing from the spirit or scope of the inventive subject matter set forth in the specification and claims. In some instances, in methodologies directly or indirectly set forth herein, various steps and operations are described in one possible order of operation, but those skilled in the art will recognize that steps and operations may be rearranged, replaced, or eliminated without necessarily departing from the spirit and scope of the present disclosure. It is intended that all matter contained in the above description or shown in the accompanying drawings shall be interpreted as illustrative only and not limiting. Changes in detail or structure may be made without departing from the spirit of the disclosure as defined in the appended claims.

1. A method of forming and monitoring a three-dimensional (3D) immune-tissue cell culture, the method comprising:
obtaining a tissue sample and a blood sample from a patient, wherein the tissue is selected from either or both of a tumor and healthy tissue;
staining isolated tissue-derived cells from the tissue sample;
staining immune cells from peripheral blood mononuclear cells (PBMCs) isolated from the blood sample;
combining the tissue-derived cells and a hydrogel to form a tissue-derived cell-containing hydrogel;
commencing, within one hour of staining the isolated tissue-derived cells, to culture the tissue-derived cell-containing hydrogel;
adding, within 24 hours of commencing to culture, the immune cells to the tissue-derived cell-containing hydrogel to form a 3D immune-tissue cell culture;
adding a test agent; and
monitoring the 3D immune-tissue cell culture over time by measuring at least two effects of the test agent on the 3D immune-tissue cell culture, the effects selected from number of the immune cells, death of the immune cells, interactions between immune cells, immune cell infiltration of the tissue-derived cell-containing hydrogel, immune cell engagement of the tissue-derived cells, immune cell killing of the tissue-derived cells, immune cell serial killing of the tissue-derived cells, death of the tissue-derived cells, and exhaustion of the immune cells,
wherein the at least two effects are measurable within 48 hours of adding the test agent.

2. The method of claim 1, wherein the monitoring is performed by live-cell microscopy selected from confocal, widefield, lightsheet, and multi-photon microscopy.

3. The method of claim 1, wherein the measuring is performed while maintaining the immune-tissue cell culture as an intact 3D immune-tissue cell culture, such that the culture is not damaged or inactivated by the measuring and/or wherein the measuring is performed on each immune cell and each tissue-derived cell.

4. (canceled)

5. (canceled)

6. (canceled)

7. The method of claim 1, wherein the monitoring comprises measuring at least one of dye fluorescence from the immune cell, dye fluorescence from the tissue-derived cell, pixel or voxel size of the immune cell, pixel or voxel size of the tissue-derived cell, pixel or voxel size of a group of immune and/or tissue-derived cells, xyz location coordinates of the immune cell, xyz location coordinates of the tissue-derived cell, speed of the immune cell, speed of the tissue-derived cell, velocity of the immune cell, and velocity of the tissue-derived cell.

8. The method of claim 1, wherein measuring immune cell infiltration comprises at least one of counting a number of the immune cells within the tissue-derived cell-containing hydrogel or calculating a distance in at least one of the x, y, and z direction traveled by the immune cells over time.

9. (canceled)

10. The method of claim 1, wherein measuring engagement of the tissue-derived cells comprises counting tissue-immune cell contact events; and/or wherein measuring serial killing of the tissue-derived cells comprises counting tissue-derived cell death events.

11. (canceled)

12. The method of claim 1, wherein measuring exhaustion of the immune cells comprises at least one of calculating a speed traveled by the immune cells and measuring a level of at least one soluble factor, wherein the soluble factor includes a cytokine, chemokine, or growth factor.

13. (canceled)

14. (canceled)

15. The method of claim 1, wherein measuring death of the immune cells comprises counting the number of instances of co-localization between the immune cells and a dye that stains dead cells and/or wherein measuring interactions between immune cells comprises counting the number of instances of contact between at least two immune cells.

16. (canceled)

17. The method of claim 1, wherein a difference between at least one of the at least two effects of the test agent on the 3D immune-tissue cell culture and the same one of the at least two effects of a control agent on the 3D immune-tissue cell culture is quantifiable within 48 hours of adding the test agent.

18. (canceled)

19. The method of claim 17, wherein the at least one effect is selected from immune cell infiltration of the tissue-derived cell-containing hydrogel, immune cell engagement of the tissue-derived cells, immune cell killing of the tissue derived cells, immune cell serial killing of the tissue derived cells, and exhaustion of the immune cells and the difference is quantifiable without disrupting the 3D immune-tissue cell culture.

20. The method of claim 17, wherein the at least one effect is selected from immune cell infiltration of the tissue-derived cell-containing hydrogel, immune cell engagement of the tissue-derived cells, and immune cell serial killing of the tissue-derived cells and the difference is quantifiable at least twice as quickly as measuring said at least one effect in an animal model of cancer comprising immunodeficient mice reconstituted with a human immune system.

21. (canceled)

22. (canceled)

23. (canceled)

24. (canceled)

25. (canceled)

26. (canceled)

27. (canceled)

28. The method of claim 1, wherein the immune cells comprise at least a first immune cell fraction and a second immune cell fraction, and the first immune cell fraction is stained with a dye that produces a color different from the second immune cell fraction, and wherein at least the first immune cell fraction or the second immune cell fraction is activated, and the activation is done by exposing the cells to at least one of a T cell activating agent, a lipopolysaccharide, a cytokine, or a colony stimulating factor.

29. (canceled)

30. The method of claim 28, wherein at least the first immune cell fraction or the second immune cell fraction comprises at least one of CD8 positive cells, CD14 positive cells, CD56 positive cells and CD8 negative cells.

31. (canceled)

32. The method of claim 1, wherein the tissue-derived cells are stained with a cell membrane permeable dye, and the cell membrane permeable dye stains at least one of lipids, proteins, organelles, cytoplasm, nuclei, and DNA.

33. (canceled)

34. The method of claim 1, wherein the tissue-derived cells are stained with a cell membrane impermeable dye, and the cell membrane impermeable dye stains DNA.

35. (canceled)

36. (canceled)

37. (canceled)

38. The method of claim 1, wherein the test agent is a cell therapy and the cell therapy is selected from lymphocyte-based therapy and myeloid-based therapy, and wherein the lymphocyte-based therapy is selected from a T-cell receptor therapy and a chimeric antigen receptor (CAR) T-cell therapy.

39. (canceled)

40. The method of claim 1, wherein the cell culture comprises a ratio of from 1 tissue to 1 immune cell to 1 tissue to 100 immune cells.

41. The method of claim 1, wherein the immune cells are added in a solid or liquid medium around the hydrogel, or the immune cells are added in a suspension to an exposed surface of the hydrogel.

42. (canceled)

43. The method of claim 1, further comprising terminating the culture and running an endpoint assay or extracting at least one of DNA, RNA, and proteins.
